SEQUENCE LISTING (7 sequences)

SEQ ID NO: 1 - DNA, 2263 bp, Homo sapiens
  misc_feature 1..2263: note = Sequence of human FAP
  source 1..2263: mol_type = genomic DNA
atggattggacctggatcctgttcctggtggctgccgctacaagggtgcactccctcaggccctccagggtccacaactccgaagagaatactatgagggccctcacactgaaggatatcctgaacggcacattctcctataaaaccttctttccaaattggatcagcggccaagagtacctccaccagagcgccgacaataacatcgtgctgtacaatatcgagaccggacaaagctatacaatcctctccaacagaaccatgaaatccgtcaatgcctccaattatggactctccccagacagacaattcgtctacctggagtccgactactccaagctctggagatatagctataccgccacatattacatttacgacctcagcaacggcgaattcgtgagggggaacgagctccccaggcccatccagtacctgtgttggagccccgtgggcagcaagctcgcctacgtctaccagaacaatatctatctcaagcaaaggcccggcgaccctcccttccagattacctttaatggcagagagaacaagatctttaacgggatccctgattgggtctatgaagaggaaatgctggccactaagtacgccctgtggtggagccccaacggcaagttcctggcctacgctgagtttaacgatacagacattccagtgatcgcttatagctactatggcgatgagcagtatcccagaaccatcaacatcccctaccccaaggccggcgccaagaatccagtcgtgagaattttcatcattgacacaacctaccccgcctacgtcggcccccaggaagtccctgtgcctgctatgatcgccagctccgactactatttcagctggctcacttgggtcaccgatgagagagtgtgcctccagtggctcaagagggtccagaacgtctccgtgctcagcatttgcgatttcagagaagactggcagacttgggattgtcccaaaacccaggaacacatcgaggaatccaggaccggctgggctggagggtttttcgtcagcaccccagtcttcagctacgatgctatcagctactataaaatcttttccgataaagacggctacaaacatatccactatatcaaggatacagtcgagaacgccatccagatcacatccggcaaatgggaggccattaatattttcagagtgacacaggacagcctgttttacagctccaacgagttcgaagagtatcctggcaggagaaacatttatagaatcagcatcggaagctatccaccctccaagaaatgcgtcacatgccacctgagaaaagagagatgccagtattacactgctagcttcagcgattatgccaagtattacgctctggtgtgttatgggcctggaattcccattagcactctgcacgatggcagaactgaccaggaaattaaaatcctggaggaaaacaaggaactggaaaacgccctgaagaacatccagctgcccaaagaagagatcaagaaactcgaggtggacgagattactctgtggtacaagatgattctcccaccccaatttgacagaagcaagaaatatcccctcctgatccaggtctacggcgggccctgttcccagagcgtgaggagcgtgttcgccgtgaactggatttcctacctggcctccaaggaaggaatggtgatcgccctggtggacggaagaggaaccgccttccaaggggataaactcctgtacgccgtgtatagaaaactgggggtgtacgaagtcgaggatcagatcactgccgtgaggaagtttatcgaaatgggcttcattgacgagaagaggatcgccatttggggctgggcttacggaggctacgtgtccagcctcgctctggctagcggcaccgggctctttaagtgcgggatcgccgtggcccccgtctccagctgggagtattacgctagcgtgtacacagaaagattcatgggcctgccaacaaaagatgacaatctggaacactataagaatagcactgtcatggctagggctgagtactttaggaacgtcgattatctcctgattcacggcactgctgacgataacgtgcacttccagaactccgcccagattgctaaagctctcgtgaacgctcaggtggacttccaggctatgtggtactccgaccaaaatcatgggctcagcggactcagcacaaaccacctgtatacccacatgactcattttctgaaacaatgtttttccctgagcgactaaa

SEQ ID NO: 2 - DNA, 2260 bp, unidentified
  misc_feature 1..2260: note = Sequence of chicken FAP
  source 1..2260: mol_type = unassigned DNA
atggattggacatggattctgttcctggtggccgctgccaccagagtgcacagcctcctgccctccaaagtcgtgacaactgtggacggcccaagggctctcaccctcgatgactatctcaacggaaactttcagtacaagacatatttcccctattgggtctccgattccgaatacctccaccagaaccaggaagatgacattatcctcttcaatgtggacatgaattacctcactaccatcatgaccaactccaccatgaagcaggtgaacgccagcaattacgtgatgagctccgacaagtattttatcgctctggaaagcaattattccaagctctggagatatagctacaccgccagctatcacatttatgatctcatctacggaggcttcgtgaccgaaaatcagctgccccacaaaattcagtacatttcctggagccccgtcggccacaagctggcttacgtctaccagaacaatatctacctcaaacaaagccccagagaggccccaattaaactcacctccgacggcaaagaaaatgaaatctttaacggaatccctgattgggtgtacgaagaggaaatgctggctaccaaatacgccctctggtggagcccaagcgggaaatacctcgcctacgtgcagtttaacgactccgatattccagtgattgagtattcctacttcggagaggaccagtaccccagaaaaattatcattccatatcctaaggccggagctaagaaccctaccgtgaaagtgttcatcgtcgacactacaaacatcgaagcctttggccctaaagaagtccctgtgccagccgtgatcgcttccagcgaccactatttcacctggctgacctgggtgaccgactccagagtcggcgtgcaatggctgaagaggattcagaacttctccgtcctggccatctgcgacttcaaagaaaacagcaacacatgggactgtcccgaaaaccagcaacatattgaagagtcccaaacaggctgggctggagggtttttcgtctccgccccatattttacatccgatggcagctcctattacaagatcttcagcgacaagaacgggtataaacacatccactacatcaatggcagcgtcgaaaacgccatccagattaccagcggcgaatgggaagccatttacattttcagagtgaccaacgatgccattttctatagctccaacgaatttgaggggtatcccggcaggagaaacatctacaaaattagcatcggatccaagccaattagaaagctgtgcatcacttgtaatctcaggaaggaaaggtgtcagtattacaccgccagattcagcgagaggagcaagtattacgccctgatctgctacgggcctggcattcctatcagcaccctgtttgagacagaaagcgacagagaactcagaatcctggaagacaaccaggagctccagtccgccctgcaagagatcattctgcccaaggaagagattaacaaactcgaggtggacggaatcaccctgtggtataagatgctgattccccctcaattcgataggagcaaaaagtacccactgctcatccaggtgtatgggggcccatgctcccagaatgtcaagcacacattcagcatcagctggattacatacctggccagcaaagaagggattatcgtcgccctggtggacggaagaggcaccgcctaccagggagataagatcctgcacgccgtctatagaaggctcggggtgtacgaagtcgaggaccaaatttccgccgtgaagaaattcattgagatgggcttcattgatgaaaagagaatcgctatctggggctgggcttatgggggctatgtcacctccctggccctgggcagcgggtccggcgtgttcaaatgcggcatcgctgtggcccccgtgtccagctgggagtactatgccagcatctatactgaaaggtttatgggcctccccgtggagagcgataacctggaacactataaaaacagcacagtgatggctagagccaaaaacttccaaaatgtggaatacctcctgatccacgggactgccgatgacaacgtccacttccaaaatagcgcccagatcgccaaggccctggtcaacgctcaggtggactttcaagccatgtggtacacagatcagaaccacggcattcccgggctcagctccaagcatctctatacccacatgactcacttcctgaagcagtgcttcagcctgagcgaataaa

SEQ ID NO: 3 - DNA, 2263 bp, unidentified
  misc_feature 1..2263: note = Sequence of Consensus FAP
  source 1..2263: mol_type = unassigned DNA
atggattggacctggattctctttctggtcgccgctgccactagagtgcacagcctcaggcctagcagggtgcacaacagcgaagggaacaccactagggccctcaccctgaaggacatcctgaatgggacattcagctacaagaccttctttcctaattggatcagcggccaggagtatctccaccaatccaccgacaataacatcgtgctctacaatattgagaccggggaatcctataccattctcagcaactccacaatgaaatccgtgaacgccagcaattacggactgtcccccgacaggcaattcgcttatctggagtccgactactccaagctgtggaggtatagctatactgccacttaccacatttatgatctgtccaatggcgaatttgtgagagggaacgagctgcccagaccaatccagtacctgtgctggagcccagtggggagcaagctcgcttacgtctaccagaacaatatttatctgaaacagagacccgaagacccaccctttcagatcacctataatggcagggagaacaagatttttaacggcatccccgactgggtgtacgaagaggaaatgctggccaccaaatacgccctctggtggtcccctaacggcaaattcctggcttatgccgagttcaacgataccgacatccctgtcatcgcctatagctattacggagacgagcagtaccctagaaccatcaatattccctacccaaaagccggcgccaaaaaccccgtcgtgaggatctttattatcgacaccacatacccagagcacgtgggacccagggaggtccccgtgcctgctatgattgcctccagcgactactatttttcctggctcacctgggtgaccgacgaaagggtgtgcctgcagtggctgaaaaggatccagaacgtgtccgtgctcagcatctgcgatttcagggaggattggcagacctgggattgtcctaagacacaggaacacattgaagagagcagaacaggctgggccggcggattctttgtgtccacccccgtgttttcctatgacgctattagctattacaaaatcttctccgataaggatggctataaacatattcactacatcaaggacactgtggagaatgccattcagattactagcggcaagtgggaggccattaacatcttcagagtcacccaggatagcctgttctactccagcaacgagtttgagggataccccggcaggagaaacatctacaggattagcatcggcagctatcccccatccaagaaatgcatcacatgtcatctgaggaaggagagatgccagtactatacagccagcttctccgactatgctaaatattacgccctggtgtgttatggccctggcctcccaatctccactctgcatgacgggagaactgaccaagagatcaagattctggaggaaaacaaggagctggaaaacgctctgaagaatatccaactgcccaaggaagagatcaagaaactggaagtggatgaaattaccctgtggtacaaaatgatcctgcctccccaattcgatagatccaaaaagtaccccctcctgatccaagtgtacgggggcccttgcagccagtccgtgaggtccgtgtttgccgtcaactggatctcctacctggccagcaaggaagggatcgtgattgccctggtggatggcagaggcacagctttccagggagataaactcctgtacgccgtctatagaaaactcggcgtgtacgaggtggaggaccagattaccgccgtgaggaagtttatcgagatgggcttcatcgatgagaaaaggatcgccatttgggggtgggcttacggagggtacgtgtccagcctcgccctggccagcggaacagggctgttcaagtgcgggatcgccgtcgcccctgtgagctcctgggaatattacgcctccatctacaccgaaaggttcatgggcctgcccactaaggatgacaatctggagcactacaagaactccactgtcatggccagggctgagtacttcagaaatgtggactacctcctgatccatggcacagctgacgataacgtgcattttcagaacagcgcccagattgccaaagccctggtgaatgcccaggtggatttccaggccatgtggtatagcgaccagaaccacggaatcagcggcctcagcaccaaacacctgtatacccatatgacccacttcctgaagcagtgtttttccctgagcgattaaa

SEQ ID NO: 4 - DNA, 19 bp, synthetic construct
  misc_feature 1..19: note = Synthetic: Feline gapdh forward primer
  source 1..19: mol_type = other DNA
aaggtgtgaaggtcggagt

SEQ ID NO: 5 - DNA, 22 bp, synthetic construct
  misc_feature 1..22: note = Synthetic: Feline gapdh reverse primer
  source 1..22: mol_type = other DNA
gatgacaagtttcccgttctca

SEQ ID NO: 6 - DNA, 23 bp, synthetic construct
  misc_feature 1..23: note = Synthetic:
  misc_feature 1..23: note = Feline FAP forward primer
  source 1..23: mol_type = other DNA
gaagttgaggaccagatcacagc

SEQ ID NO: 7 - DNA, 21 bp, synthetic construct
  misc_feature 1..21: note = Synthetic:
  misc_feature 1..21: note = Feline FAP reverse primer
  source 1..21: mol_type = other DNA
catagcctccgtaggaccagc

(19) United States
(12) Patent Application Publication
(10) Pub. No.: US 2025/0002998 A1
(43) Pub. Date: Jan. 2, 2025

(54) URINE PCR ASSAY AND USE THEREOF TO DIAGNOSE AND STAGE FELINE CHRONIC KIDNEY DISEASE

(71) Applicant: EDGE ANIMAL HEALTH, INC., Boston, MA (US)

(72) Inventors: Michelle Lynn HAVEN, Hampton, NJ (US); Kristi Mae MOORE, Shawnee, KS (US); Richard E. PETERSON, Jr., Ijamsville, MD (US)

(73) Assignee: EDGE ANIMAL HEALTH, INC., Boston, MA (US)

(21) Appl. No.: 18/468,113

(22) Filed: Sep. 15, 2023

Related U.S. Application Data
(60) Provisional application No. 63/510,942, filed on Jun. 29, 2023.

(51) Int. Cl. C12Q 1/6883

(52) U.S. Cl. CPC C12Q 1/6883; C12Q 2600/112; C12Q 2600/158

(57) ABSTRACT

This invention is related, generally, to a novel assay for Feline Chronic Kidney Disease (CDK) and, more particularly, to detecting the presence of a target compound in a sample which may contain the target compound, amplification, and subsequent quantitation of the detector molecule to permit both the diagnosis and staging of feline CDK. The present assay is particularly useful for diagnosing early CDK, when clinical signs may not be present, to permit intervention and treatment. Another embodiment provides target compounds, such as antibodies, and methods using these compounds for the treatment of renal fibrosis associated with chronic kidney disease. In one embodiment, the present invention provides a pharmaceutical composition comprising a mRNA encoding a chimeric antigen receptor (CAR) antibody, and a transfer vehicle comprising a lipid nanoparticle (LNP), and methods of producing CAR-T cells that limit the progression of renal disease in mammal.

### CROSS-REFERNECE TO RELATED APPLICATIONS

This application claims the benefit of priority to U.S. Provisional Application No. 63/367,207, filed Jun. 28, 2022, and to U.S. Provisional Application No. 63/510,942, filed Jun. 29, 2023, each of which is incorporated herein in their entirety by reference.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 29, 2023, is named “067443.005US1.xml” and is 17,519 bytes in size. The sequence listing contained in this .XML file is part of the specification and is hereby incorporated by reference herein in its entirety.

### TECHNICAL FIELD OF THE INVENTION

This invention is related, generally, to a novel assay for Feline Chronic Kidney Disease (CDK) and, more particularly, to detecting the presence of a target compound in a sample which may contain the target compound, using a nucleic acid detector molecule, amplification and subsequent quantitation of the detector molecule to permit both the diagnosis and staging of feline CDK, especially in early CDK when clinical signs may not be present. In another embodiment of the present invention, target compounds of the present invention are useful in developing compositions, such as antibodies, and methods for the treatment of renal fibrosis associated with chronic kidney disease, especially feline CDK.

### BACKGROUND OF THE INVENTION

Feline chronic kidney disease (CKD), a progressive and irreversible condition that affects the kidneys of cats, is a common disease in older cats and can lead to serious health complications if left untreated. Interestingly, when data in dogs and cats were similarly obtained, the prevalence of CKD in geriatric cats exceeded that observed in geriatric dogs by 2-fold or more. The prevalence increases in cats from 5 to 6 years onward, with estimates of CKD in geriatric cats ranging from 35% to 81%. For example, a recent retrospective study by the Royal Veterinary College (RVC) reported that about 30% of cats aged 10 years or older have CKD, which equates to approximately 600,000 cases alone in the UK. Of these, about 40% will also have hypertension. Other studies report that CKD may affect as many as 50% of elderly cats, with prevalence increasing with age (Marino, C. L. et al, J Feline Med Surg., 16(6):465-472 (2013)).

A second trend is the increasing prevalence of the diagnosis of CKD in cats during recent decades. Data from the Purdue Veterinary Medical Database suggests that the overall prevalence of feline CKD increased from 0.04% in the 1980s to 0.2% in 1990s to 1% by the 2000s (Brown, C. A., et al., Vet Pathol., 53(2):309-236 (2016)). Whether this increase is a reflection of increased awareness with enhanced diagnostic acumen, an increase in the median age within cat populations, or a true increase in prevalence is unknown.

The diagnosis of feline CKD is based on a combination of clinical signs, laboratory tests, and imaging studies. The most common clinical signs of CKD in cats include increased thirst and urination, weight loss, poor appetite, vomiting, and lethargy. Laboratory tests such as blood chemistry, urinalysis, and urine culture can help to confirm the diagnosis of CKD. Imaging studies such as ultrasound can also be used to evaluate the kidneys and assess the severity of the disease.

Feline CKD is categorized into 4 stages based on, amongst other signs, creatinine levels and pathological changes in the kidney associated with each stage have been described. stage I or early kidney disease is characterized as associated with mild kidney damage and no clinical signs, although lesions that involve as little as 25%-50% of the parenchyma can impact function. Stage 2 is classified as mild to moderate kidney disease, with mild clinical signs, while Stage 3 is described as moderate to severe kidney disease and Stage 4 characterized as end-stage kidney disease with severe clinical signs. Although progressive fibrosis is considered to be a hallmark of the disease, there appears to be no significant difference in the percentage of fibrosis between stage II and stage III, leading to the hypothesis that factors other than fibrosis may contribute to later stage disease progression.

Currently, treatment of CKD includes managing clinical signs and attempting to slow progression of the disease. Treatment options include providing a special diet low in protein and phosphorus to help reduce kidney workload and prescribing medications, such as ACE inhibitors, phosphate binders, and erythropoietin, to help to manage the clinical signs and improve the cat's quality of life. Fluid replacement therapy can be provided as an adjunct to other treatments to maintain hydration and improve kidney function. Regular monitoring of the cat's kidney function and clinical signs is essential to assess the effectiveness of the treatment, while complications such as anemia, hypertension, and urinary tract infections should be managed promptly to prevent further damage to the kidneys. Life expectancy of a cat with CKD depends on several factors, including the stage of the disease, the cat's age, and the presence of other medical conditions. However, in general, cats with CKD have a reduced life expectancy compared to healthy cats, but with appropriate treatment and management, many cats with CKD can live for several years.

The exact cause of feline CKD is not fully understood, but there are several factors that can contribute to the development of the disease, including age because not only is CKD more common in older cats, but the risk of developing the disease also increases with age. Genetics is also thought to play a role because certain breeds, such as Persians and Siamese cats, are more prone to developing the disease. Chronic infections of the urinary tract or kidneys can lead to kidney damage and subsequently to the development of CKD as can exposure to certain toxins, such as antifreeze, which can cause kidney damage. Other medical conditions, especially those associated with damage to kidney function such as diabetes, hyperthyroidism, and hypertension, are known to increase the risk of a cat developing CKD. However, it is important to note that in many cases the exact cause of feline CKD is unknown, and the disease may develop as a result of a combination of factors.

Fibrosis, a common feature of feline CKD, is a process in which excess connective tissue is deposited in the kidneys leading to scarring and loss of function. Thus, fibrosis and fibrosis activating factor (FAF), a protein that has been identified as a potential contributor to the development of fibrosis in the kidneys, is thought to play a significant role in the progression of the disease. FAF, produced by kidney cells in response to injury or inflammation, stimulates the production of fibroblasts which then produce connective tissue. Studies have shown that FAF levels are elevated in cats with CKD, and that higher levels of FAF are associated with more severe fibrosis and kidney damage, supporting the role FAF may have in the development and progression of fibrosis in feline CKD. Although research is ongoing to better understand the role of FAF, as well as to develop treatments that target this protein as a way to slow or prevent the progression of the disease, there currently are no such specific treatments.

There is also great interest amongst researchers and clinicians to diagnose early stage CDK because intervention then may be most useful for decreasing mortality and morbidity associated with later stage CDK. Unfortunately, current diagnosis relies upon identification of clinical signs, laboratory testing, and imaging results and none are able to quickly and accurately stage the disease until progression is quite far along. For example, IDEXX promotes their SDMA test as being able to diagnose CKD at an earlier stage than when relying on creatinine levels, when there has been 20%-40% loss of kidney function (prior to stage IV).

Previously, urine has been used as a sample from which to isolate particular mRNA species as a biomarker in humans to detect both renal fibrosis as well as other renal pathophysiological conditions (Chun-Yan et al, Liquid biopsy biomarkers of renal interstitial fibrosis based on urinary exosome, Exp Mol Pathol 105:223-228, 2018). Thus, the present invention has found, surprisingly, that urine can be used as a sample for a rapid and reliable assay in cats not only to diagnose CDK, but to actually stage the disease especially in early cases before clinical signs may be present.

Thus, it is an object of the invention to provide a reliable and quick diagnostic assay for feline Chronic Kidney Disease, especially one that can help to accurately stage the disease even before clinical signs are useful.

### SUMMARY OF THE INVENTION

In accordance with the purpose(s) of this invention, as embodied and broadly described herein, this invention in one aspect is directed to compositions and methods relating to an assay for the diagnosis and staging of Feline Chronic Kidney Disease (CDK) that is rapid, reliable, and can be used for detecting the presence of a target indicator in body fluids such as urine. In one embodiment, the assay used is a polymerase chain reaction (PCR). Another embodiment of the present invention uses reverse transcription PCR (RT-PCR) in an assay to diagnose and stage Feline CDK.

PCR is a primer extension reaction that provides a method to amplify a specific DNA or polynucleotide in vitro, generating thousands to millions of copies of a particular DNA sequence. PCR is now a common and often indispensable technique used in medical and biological research labs for a variety of applications including, but not limited to DNA cloning for sequencing, functional analysis of genes; rapid disease diagnosis; the identification of genetic fingerprints; and the like. In addition to RT-PCR, there are a number of variations on the basic PCR test including, amongst others, quantitative real-time PCR (qPCR or RT-PCR), allele specific PCR, asymmetric PCR, hot start PCR, multiplex-PCR, nested-PCR, ligation-mediated PCR, intersequence-specific PCR, thermal asymmetric interlaced PCR and touchdown-PCR. PCR assays and variations thereof provide a wide variety of uses for different purposes. For example, single-nucleotide polymorphisms (SNPs) (single-base differences in DNA) can be identified by allele-specific PCR, whilst qPCR can provide a high degree of precision in determining the number of copies amplified in the PCR reactions

Unlike PCR, wherein a specific DNA is amplified, RT-PCR amplifies RNA targets by converting the RNA template into complementary (c) DNA using reverse transcriptase. The cDNA serves later as a template for exponential amplification. (see, Bartlett et al., “A Short History of the Polymerase Chain Reaction”, PCR Protocols, 2003).

RT-qPCR, or quantitative reverse transcription PCR, combines the effects of reverse transcription and quantitative PCR or real-time PCR to amplify, detect, and quantify a specific target. The process is performed by reverse transcription of total RNA or mRNA to complementary DNA (cDNA) using reverse transcriptase, followed by amplification and detection of specific targets of this cDNA using quantitative PCR (qPCR) or real-time PCR. At each cycle during this PCR, the quantity of DNA can be measures in real-time by using a variety of fluorescent chemistries, including by using either hydrolysis probes such as TaqMan® probes, or a double-stranded DNA binding dye such as SYBR® Green dye. RT-qPCR has a variety of applications including quantifying gene expression levels, validating RNA interference (RNAi), and detecting pathogens such as viruses. The selection of fluorescent chemistry depends upon a variety of factors such as the application, cost, and whether the assay is a singleplex or multiplex assay. DNA-binding dyes are preferred for singleplex, low-throughput assays since they are easier to design, have lesser set-up time, and are more cost-efficient. Fluorescent probes are more commonly employed in high-throughput, multiplex assays that require higher specificity.

In order to detect fibroblast activation protein (FAP) mRNA, the present invention contemplates using urine as the sample in the rapid assay. Urine can be collected from cats using standard methods well known to those in the art, although the optimal collection methods will provide for sterile collection. For example, urine collection may be facilitated by catheterization or, alternately, urine samples may be collected from cats via cystocentesis in the morning prior to void when the bladder is full. Cystocentesis removes urine directly from the bladder using a needle so that the sample is fresh and without the risk of contamination when passing through the urethra.

In one embodiment, the present invention provides compositions, methods, and kits for amplifying and/or detecting FAP mRNA isolated from a feline urine sample. The process begins by isolating mRNA from the urine sample and then, using reverse transcription, producing cDNA for subsequent amplification and detection of specific targets using quantitative PCR (qPCR) or real-time PCR. The present invention also contemplates various oligonucleotides useful in the assay, each oligonucleotide recognizing a target sequence within a FAP target region or its complementary sequence. In alternate embodiments, one of more of the oligonucleotides may serve as amplification oligomers and/or detection probes for amplification and/or detection of corresponding FAP target nucleic acid. An amplification oligomer is configured to specifically hybridize to a target sequence within a target nucleic acid. At least two amplification oligomers flanking a target region within the target nucleic acid are utilized in an in vitro nucleic acid amplification reaction to generate an amplicon therefrom. Exemplary in vitro amplification reactions include, for example, 10 PCR (e.g., Taqman® PCR) and transcription-associated amplification (e.g., TMA or NASBA). A detection probe, configured to specifically hybridize to a target sequence flanked by at least two amplification oligomers, may be utilized to hybridize specifically to at least a portion of an amplification product, either after completion of or during the amplification process. Methods of the present invention may further may use an oligonucleotide that serves as a capture probe for processing a sample by capturing a FAP target nucleic acid and separating it from other sample components (see, e.g., U.S. Pat. Nos. 6,110,678, 6,280,952, and 6,534,273, each of which is incorporated by reference herein in its entirety).

In another aspect, the invention relates to a method for differentially staging feline CKD, particularly by identifying early CDK even before clinical signs may be present, to provide the owner with the ability to provide supportive care that may ameliorate or slow progression of the disease before significant renal damage occurs. The present invention contemplates identifying early stage disease and differentiating it from later stages by quantifying the amount of FAP target nucleic acid.

In certain embodiments, oligonucleotides and methods of the present invention are useful for amplifying and detecting nucleic acid target sequences present in a sample in a relatively short time so that diagnosis can be made quickly and, in a preferred embodiment provide an assay able to detect early stage feline kidney disease so that effective treatment can be initiated to limit and perhaps improve the outcome for the cat.

The present invention also contemplates methods for detecting a FAP nucleic acid which, optionally, include a detecting step that uses at least one probe that specifically hybridizes to the FAP amplification product (RNA, DNA amplicon, or cDNA). Accordingly, in certain embodiments, a detection probe of the present invention is configured to specifically hybridize to a region within a target nucleic acid selected from FAP nucleic acid. In certain embodiments, a set of oligonucleotides for detection of FAP mRNA includes two or more detection probes selected from the probes above, whereby the probes are for detecting two or more regions of a FAP target nucleic acid region. In certain embodiments, a detection probe is configured to specifically hybridize to a target nucleic acid region selected from a region within a FAP nucleic acid sequence. In particular variations, a detection probe for detecting an FAP target nucleic acid region is configured to specifically hybridize to a region corresponding to to a specific region. In some variations, a set of oligonucleotides for detecting FAP target nucleic acid regions includes two or more detection probes selected from the probes above, where the probes are for detecting two or more of an FAP target nucleic acid region.

In particular embodiments, a detection probe as above-configured to specifically hybridize to a target nucleic acid region selected from (a) a region within a FAP nucleic acid sequence corresponding to a specific region. Although these sequences are shown as cDNA sequences, equivalent RNA or RNA/DNA chimeric sequences can be readily derived by the person skilled in the art and are to be considered as falling within the definition of “oligomer” or “detection probe.” In addition, complementary sequences of DNA and RNA and reverse complementary sequences can be readily derived by the skilled person. It is therefore to be understood that a description of any individual sequence of DNA, for example, encompasses its complement, its reverse complement, and equivalent RNA or RNA/DNA chimeric sequences.

Typically, a detection probe in accordance with the pres-ent invention further includes a label. Particularly suitable labels include compounds that emit a detectable light signal, e.g., fluorophores or luminescent (e.g., chemiluminescent) compounds that can be detected in a homogeneous mixture. More than one label, and more than one type of label, may be present on a particular probe, or detection may rely on using a mixture of probes in which each probe is labeled with a compound that produces a detectable signal (see, e.g., U.S. Pat. Nos. 6,180,340 and 6,350,579, each incorporated by reference herein in its entirety). Labels may be attached to a probe by various means including covalent linkages, chelation, and ionic interactions, but preferably the label is covalently attached. For example, in some embodiments, a detection probe has an attached chemiluminescent label such as, e.g., an acridinium ester (AE) compound (see, e.g., U.S. Pat. Nos. 5,185,439; 5,639,604; 5,585,481; and 5,656,744; each incorporated by reference herein), which in typical variations is attached to the probe by a non-nucleotide linker (see, e.g., U.S. Pat. Nos. 5,585,481; 5,656,744; and 5,639, 604, particularly at column 10, line 6 to column 11, line 3, and Example 8; each incorporated by reference herein in its entirety). In other embodiments, a detection probe comprises both a fluorescent label and a quencher, a combination that is particularly useful in fluorescence resonance energy trans-fer (FRET) assays. Specific variations of such detection probes include, e.g., a TaqMan detection probe (Roche Molecular Diagnostics) and a “molecular beacon” (see, e.g., yagi et al., Nature Biotechnol. 16:49-53, 1998; U.S. Pat. Nos. 5,118,801 and 5,312,728; each incorporated by refer-ence herein in its entirety).

A detection probe may further include a non-target-hybridizing sequence. Specific embodiments of such detection probes include, for example, probes that form conformations held by intramolecular hybridization, such as conformations generally referred to as hairpins. Particularly suitable hairpin probes include a “molecular torch” (see, e.g., U.S. Pat. Nos. 6,849,412; 6,835,542; 6,534,274; and 6,361,945, each incorporated by reference herein in its entirety) and a “molecular beacon” (see, e.g., Tyagi et al., supra; U.S. Pat. Nos. 5,118,801 and 5,312,728, supra). Methods for using such hairpin probes are well known in the art.

In particular embodiments, each of one or more detection probes for detecting one or more FAP amplification products includes a fluorescent label (“fluorescent dye compound”). Suitable fluorophores are well-known in the art and include, for example, CalO 560, CalRed 610, and FAM. In some variations of an oligonucleotide set for determining the presence or absence of FAP in the sample, at least one FAP specific detection probe is labeled with a different fluorophore.

In some such embodiments comprising fluorophore-labeled detection probes, the detection probe(s) further include a quencher. Suitable quenchers are well-known in the art and include, for example, BHQ, TAMRA, and DABCLY. A method for determining the presence or absence of FAP, in accordance with the present invention, generally includes the following steps: (1) contacting a sample suspected of containing FAP with at least two amplification oligomers as described above for amplification of the FAP target nucleic acid region; (2) performing an in vitro nucleic acid amplification reaction, where any FAP target nucleic acid, if present in the sample, is used as a template for generating one or more amplification products corresponding to the target nucleic acid present in the sample; and (3) either (i) determining the sequences of the one or more amplification products or (ii) detecting the presence or absence of the one or more amplification products using one or more detection probes as described above for the FAP target nucleic acid regions.

One embodiment of a method according to the present invention generally comprises (a) extracting FAP mRNA from the sample obtained from the subject; (b) using reverse transcriptase reaction to obtain cDNA template; (c) adding the cDNA, a positive control, and optionally a negative control into PCR tubes of PCR reaction system respectively to obtain a corresponding sample reaction tube, positive reaction tube or negative reaction tube, wherein the PCR reaction system contains the primers for detecting FAP (d) performing PCR reaction by placing the reaction tubes on a PCR instrument, setting circulation parameters, and performing PCR reaction; (d) analyzing the results after the PCR reaction is completed; and (e) determining the presence of FAP in the sample; wherein the presence of FAP indicates the presence of fibrosis that precedes renal disease.

In yet another embodiment, a Method according to the present invention differentiates between stages of CKD, especially in early stages where clinical signs are not evident, by quantifying the amount of FAP present in a sample, wherein the presence of FAP correlates with the presence of fibrosis.

In yet another aspect, the invention relates to a PCR diagnostic assay kit for the rapid and reliable diagnosis of CDK in a feline subject comprising (a) primers for detecting FAP from a mRNA sample comprising a forward primer having a nucleotide sequence as set forth in SEQ ID NO:6 and a reverse primer having a nucleotide sequence as set forth in SEQ ID NO:7 (b) DNA polymerase; (c) blocking oligonucleotides; (d) a detectable label; and (e) buffers.

In yet another aspect, the invention relates to a PCR diagnostic assay kit for the rapid and reliable diagnosis of CDK in a feline subject comprising (a) primers for detecting Glyceraldehyde 3-phosphate dehydrogenase (GAPDH) from a mRNA sample comprising a forward primer having a nucleotide sequence as set forth in SEQ ID NO: 4 and a reverse primer having a nucleotide sequence as set forth in SEQ ID NO:5 (b) DNA polymerase; (c) blocking oligonucleotides; (d) a detectable label; and (e) buffers. The quantification of GAPDH will be used and a normalization between samples.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIGS. 1A-1B are illustrative of the immunoliposomes used for targeted therapy.

FIG. 2 is illustrative of the structure of CAR.

FIG. 3 is illustrative of the use of mRNA/LNP to deliver transient in vivo CAR-T cell to treat feline Chronic Kidney Disease (CKD).

FIG. 4 is illustrative of immunoliposomes as a game changing platform for targeted therapeutic development.

FIG. 5 is illustrative of the loading of any mRNA into targeted liposomes depending on the disease being treated on a prescription basis.

FIG. 6 shows the complete 7×7 epitope binning assay.

FIGS. 7A-7F are SDS PAGE gels a time-course of feline FAP (fFAP) protein expression.

FIGS. 8A-8E demonstrates the results of immunohistochemistry staining of kidney sections from a cat (#20-10270) without fibrosis using an FAP antibody.

FIGS. 9A-9E demonstrates the results of immunohistochemistry staining of kidney sections from a cat (#20-01692) with a low level of fibrosis using an FAP antibody.

FIGS. 10A-10E demonstrates the results of immunohistochemistry staining of kidney sections from a cat (#20-00774) with a higher level of fibrosis using an FAP antibody.

FIGS. 11A-11E demonstrates the results of immunohistochemistry staining of kidney sections from a cat (#20-06613) with a higher level of fibrosis using an FAP antibody.

### DETAILED DESCRIPTION OF THE INVENTION

### I. Definitions

It is to be understood that the terminology used herein is for the purpose of describing particular embodiments only, and is not intended to be limiting. As used in the present Specification and the Claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

Unless otherwise specified, the experimental methods, detection methods, and preparation methods disclosed in the present invention all adopt the conventional molecular biology, biochemistry, microbiology, cell biology, genomics, and recombinant polynucleotides, chromatin structure and analysis, analytical chemistry, cell culture, recombinant DNA technology and related fields in the technical field. These techniques have been well described in the existing literature. For details, please refer to inter alia Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol.304, Chromatin (P M Wassarman and A P Wolfe, eds.), Academic Press, San Diego, 1999; and METHODS IN MOLECULAR BIOLOGY, Vol. 119, Chromatin Protocols (P B Becker, ed.) Humana Press, Totowa, 1999, et al.; Cellular and Molecular Immunology, Ninth Edition, A. K. Abbas., et al., Elsevier (2017), ISBN 978-0323479783; Cancer Immunotherapy Principles and Practice, First Edition, L. H. Butterfield, et al., Demos Medical (2017), ISBN 978-1620700976; Janeway's Immunobiology, Ninth Edition, Kenneth Murphy, Garland Science (2016), ISBN 978-0815345053; Clinical Immunology and Serology: A Laboratory Perspective, Fourth Edition, C. Dorresteyn Stevens, et al., F. A. Davis Company (2016), ISBN 978-0803644663; Antibodies: A Laboratory Manual, Second edition, E. A. Greenfield, Cold Spring Harbor Laboratory Press (2014), ISBN 978-1-936113-81-1; Culture of Animal Cells: A Manual of Basic Technique and Specialized Applications, Seventh Edition, R. I. Freshney, Wiley-Blackwell (2016), ISBN 978-1118873656; Transgenic Animal Technology, Third Edition: A Laboratory Handbook, C. A. Pinkert, Elsevier (2014), ISBN 978-0124104907;The Laboratory Mouse, Second Edition, H. Hedrich, Academic Press (2012), ISBN 978-0123820082; Manipulating the Mouse Embryo: A Laboratory Manual, Fourth Edition, R. Behringer, et al., Cold Spring Harbor Laboratory Press (2013), ISBN 978-1936113019; PCR 2: A Practical Approach, M. J. McPherson, et al., IRL Press (1995), ISBN 978-0199634248;Methods in Molecular Biology (Series), J. M. Walker, ISSN 1064-3745, Humana Press; RNA: A Laboratory Manual, D. C. Rio, et al., Cold Spring Harbor Laboratory Press (2010), ISBN 978-0879698911; Methods in Enzymology (Series), Academic Press; Molecular Cloning: A Laboratory Manual (Fourth Edition), M. R. Green, et al., Cold Spring Harbor Laboratory Press (2012), ISBN 978-1605500560; Bioconjugate Techniques, Third Edition, G. T. Hermanson, Academic Press (2013), ISBN 978-0123822390; Methods in Plant Biochemistry and Molecular Biology, W. V. Dashek, CRC Press (1997), ISBN 978-0849394805; Plant Cell Culture Protocols (Methods in Molecular Biology), V. M. Loyola-Vargas, et al., Humana Press (2012), ISBN 978-1617798177; Plant Transformation Technologies, C. N. Stewart, et al., Wiley-Blackwell (2011), ISBN 978-0813821955; Recombinant Proteins from Plants (Methods in Biotechnology), C. Cunningham, et al., Humana Press (2010), ISBN 978-1617370212; Plant Genomics: Methods and Protocols (Methods in Molecular Biology), W. Busch, Humana Press (2017), ISBN 978-1493970018; Plant Biotechnology: Methods in Tissue Culture and Gene Transfer, R. Keshavachandran, et al., Orient Blackswan (2008), ISBN 978-8173716164.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds, phosphorothioate bonds, disulfide bonds and peptide bonds (—CO—NH—).

“Non-covalent bond,” “non-covalently attached,” “non-covalently bound,” “non-covalently linked,” “non-covalent interaction,” and “non-covalently connected” are used interchangeably herein and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to, hydrogen bonding, ionic interactions (e.g., NaCl), van der Waals interactions, and hydrophobic bonds.

As used herein, “hydrogen bonding,” “hydrogen-base pairing,” and “hydrogen bonded” are used interchangeably and refer to canonical hydrogen bonding and non-canonical hydrogen bonding including, but not limited to, “Watson-Crick-hydrogen-bonded base pairs” (W-C-hydrogen-bonded base pairs or W-C hydrogen bonding); “Hoogsteen-hydrogen-bonded base pairs” (Hoogsteen hydrogen bonding); and “wobble-hydrogen-bonded base pairs” (wobble hydrogen bonding). W-C hydrogen bonding, including reverse W-C hydrogen bonding, refers to purine-pyrimidine base pairing, e.g., adenine: thymine, guanine: cytosine, and uracil: adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W-C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine: uracil, inosine (hypoxanthine): uracil, inosine-adenine, and inosine-cytosine. Rules for canonical hydrogen bonding and non-canonical hydrogen bonding are known to those of ordinary skill in the art (see, e.g., The RNA World, Third Edition (Cold Spring Harbor Monograph Series), R. F. Gesteland, Cold Spring Harbor Laboratory Press (2005), ISBN 978-0879697396; The RNA World, Second Edition (Cold Spring Harbor Monograph Series), R. F. Gesteland, et al., Cold Spring Harbor Laboratory Press (1999), ISBN 978-0879695613; The RNA World (Cold Spring Harbor Monograph Series), R. F. Gesteland, et al., Cold Spring Harbor Laboratory Press (1993), ISBN 978-0879694562 (see, e.g., Appendix 1: Structures of Base Pairs Involving at Least Two Hydrogen Bonds, I. Tinoco); Principles of Nucleic Acid Structure, W. Saenger, Springer International Publishing AG (1988), ISBN 978-0-387-90761-1; Principles of Nucleic Acid Structure, First Edition, S. Neidle, Academic Press (2007), ISBN 978-01236950791).

As used herein, a molecule is said to be able to “immunospecifically bind” a second molecule if such binding exhibits the specificity and affinity of an antibody to its cognate antigen. Antibodies are said to be capable of immunospecifically binding to a target region or conformation (“epitope”) of an antigen if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACORE® assays, or other assays known in the art, but would not bind to a totally unrelated antigen. In some embodiments, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

As used herein, “immunosuppressive” means that the expression of said non-endogenous polypeptide has the effect of alleviating the immune response of the patient host against the donor's immune cells.

As used herein, “autologous” means that cells, cell lines or population of cells used for treating subjects are originating from said subject or from a Human Leucocyte Antigen (HLA) compatible donor.

As used herein, “allogeneic” means that the cells or population of cells used for treating subjects are not originating from said subject but from a donor.

As used herein, a molecule is said to “physiospecifically bind” a second molecule if such binding exhibits the specificity and affinity of a receptor to its cognate binding ligand. A molecule can be capable of physiospecifically binding to more than one other molecule.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250contiguous amino acid residues.

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product(s).” Expression may include splicing the mRNA in a eukaryotic cell, if the polynucleotide is derived from genomic DNA.

As used herein, the term fluorescent detection label refers to a fluorophores useful for the detection of nucleic acids, including oligonucleotides and the like, and includes but is not limited to FAM (5′ 6-FAM (Fluorescein), which is a single isomer derivative of fluorescein that can be attached to 5′ or 3′ end of oligos; CAL Fluor Red 610-dT, a phosphoramidite used for labelling of oligonucleotides by adding a yellow-red fluorescent dye internally or to the 5′ end of an oligonucleotide; Texas Red®; FITC; LC Red460; and others that are known to those of skill in the art.

As used herein the term “modulate” relates to a capacity to alter an effect, result, or activity (e.g., signal transduction). Such modulation can be agonistic or antagonistic and can be assayed by determining any characteristic directly or indirectly affected by the expression of the target gene. Such characteristics include, for example, changes in RNA or protein levels, protein activity, product levels, expression of the gene, or activity level of reporter genes. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. In some embodiments, such modulation will provide at least a 10% change in a measurable immune system activity, at least a 50% change in such activity, or at least a 2-fold, 5-fold, 10-fold, or at least a 100-fold change in such activity.

“Vector” and “plasmid,” as used herein, refer to a polynucleotide vehicle to introduce genetic material into a cell. Vectors can be linear or circular. Vectors can contain a replication sequence capable of effecting replication of the vector in a suitable host cell (e.g., an origin of replication). Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art. The four major types of vectors are plasmids, viral vectors, cosmids, and artificial chromosomes. Typically, vectors comprise an origin of replication, a multicloning site, and/or a selectable marker.

As used herein, a “host cell” generally refers to a biological cell. A cell is the basic structural, functional, and/or biological unit of an organism. A cell can originate from any organism having one or more cells. Examples of host cells include, but are not limited to, a prokaryotic cell, eukaryotic cell, a bacterial cell, an archaeal cell, a cell of a single-cell eukaryotic organism, a cell of a eukaryotic organism, a protozoal cell, a cell from a plant (e.g., cells from plant crops (such as soy, tomatoes, sugar beets, pumpkin, hay, cannabis, tobacco, plantains, yams, sweet potatoes, cassava, potatoes, wheat, sorghum, soybean, rice, corn, maize, oil-producing Brassica (e.g., oil-producing rapeseed and canola), cotton, sugar cane, sunflower, millet, and alfalfa), fruits, vegetables, grains, seeds, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. agardh, and the like), seaweeds (e.g., kelp), a fungal cell (e.g., a yeast cell or a cell from a mushroom), an animal cell, a cell from an invertebrate animal (e.g., fruit fly, cnidarian, echinoderm, nematode, and the like), a cell from a vertebrate animal (e.g., fish, amphibian, reptile, bird, or mammal), a cell from a mammal (e.g., a pig, a cow, a goat, a sheep, a rodent, a rat, a mouse, a non-human primate, a human, and the like). Furthermore, a cell can be a stem cell or a progenitor cell. In some embodiments, a host cell is a non-human cell. In some embodiments, a host cell is a human cell outside of a human body, wherein in particular embodiments the human cell is not introduced into a human body.

As used herein, “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells can be embryonic, fetal, amniotic, adult, or induced pluripotent stem cells.

As used herein, “induced pluripotent stem cell” refers to a type of pluripotent stem cell that is artificially derived from a non-pluripotent cell, typically a somatic cell. In some embodiments, the somatic cell is a human somatic cell. Examples of somatic cells include, but are not limited to, dermal fibroblasts, bone marrow-derived mesenchymal cells, cardiac muscle cells, keratinocytes, liver cells, stomach cells, neural stem cells, lung cells, kidney cells, spleen cells, and pancreatic cells. Additional examples of somatic cells include cells of the immune system, including but not limited to, B cells, dendritic cells, granulocytes, innate lymphoid cells, megakaryocytes, monocytes/macrophages, myeloid-derived suppressor cells, natural killer (NK) cells, T cells, thymocytes, and hematopoietic stem cells.

As used herein, the “activating” or “stimulatory” signals encompass signals that result in enhancing an activity or enhancing signal transduction.

As used herein, “suppressive” signals refer to signals that suppress immune activity.

The term “endogenous concentration” refers to the level at which a molecule is natively expressed (i.e., in the absence of expression vectors or recombinant promoters) by a cell (which cell can be a normal cell, a cancer cell or an infected cell).

“Subject,” as used interchangeably with “individual,” “host,” and “patient,” refers to any member of the mammalian species, including, without limitation, humans and other primates, including non-human primates such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys and other gallinaceous birds, ducks, and geese; and the like. The term does not denote a particular age or gender. Thus, the term includes adult, young, and newborn individuals as well as male and female. In a specific embodiment of the present invention, the term “subject” refers to a feline. In some embodiments, a host cell is derived from a subject (e.g., stem cells, progenitor cells, or tissue-specific cells).

As used herein, the terms “treat,” “treating,” “treatment” and “therapeutic use” refer to the elimination, reduction or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the term “prophylactic agent” refers to an agent that can be used in the prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, an “immune cell” refers to any cell from the hemopoietic origin including, but not limited to, T cells, B cells, monocytes, dendritic cells, and macrophages.

As used herein, “inflammatory molecules” refer to molecules that result in inflammatory responses including, but not limited to, cytokines and metalloproteases such as including, but not limited to, IL-1β, TNF-α, TGF-beta, IFN-γ, IL-18, IL-17, IL-6, IL-23, IL-22, IL-21, and MMPs.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, the terms “wild-type,” “naturally occurring,” and “unmodified” refer to the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

As used herein, the terms “engineered,” “genetically engineered,” “recombinant,” “modified,” “non-naturally occurring,” “non-natural,” and “non-native” are interchangeable and indicate intentional human manipulation.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (—0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:
100 times the fraction W/Z,
   where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The terms “chimeric protein,” as used herein, refers to a single protein created by joining two or more proteins, protein domains, or protein fragments or circular permuted polypeptides that do not naturally occur together in a single protein. In some embodiments, a linker polynucleotide can be used to connect a first protein, protein domains, or protein fragments, or circular permuted polypeptides to a second protein, protein domains, or protein fragments or circular permuted polypeptides.

As used herein, “aptamer” refers to single stranded nucleic acid. Structurally, the aptamers of the present disclosure are specifically binding oligonucleotides. Aptamers may comprise RNA, DNA or both RNA and DNA. The aptamer may be synthetically produced using art known methods. Alternatively, the aptamer may be recombinantly produced.

As used herein, the term “between” is inclusive of end values in a given range (e.g., between 1 and 50 nucleotides in length includes 1 nucleotide and 50 nucleotides; between 5 amino acids and 50 amino acids in length includes 5 amino acids and 50 amino acids).

The terms “oligonucleotide” and “polynucleotide” as used interchangeably herein refer to a polymer of greater than one nucleotide in length of ribonucleic acid (RNA), deoxyribonucleic acid (DNA), hybrid RNA/DNA, modified RNA or DNA, or RNA or DNA mimetics. The polynucleotides may be single- or double-stranded. The terms include polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

The terms “primer” and “polynucleotide primer,” as used herein, refer to a short, single-stranded polynucleotide capable of hybridizing to a complementary sequence in a nucleic acid sample. A primer serves as an initiation point for template-dependent nucleic acid synthesis. Nucleotides are added to a primer by a nucleic acid polymerase in accordance with the sequence of the template nucleic acid strand. A “primer pair” or “primer set” refers to a set of primers including a 5′ upstream primer that hybridizes with the 5′ end of the sequence to be amplified and a 3′ downstream primer that hybridizes with the complementary 3′ end of the sequence to be amplified. The term “forward primer” as used herein, refers to a primer which anneals to the 5′ end of the sequence to be amplified. The term “reverse primer”, as used herein, refers to a primer which anneals to the complementary 3′ end of the sequence to be amplified.

The terms “probe” and “polynucleotide probe,” as used herein, refer to a polynucleotide used for detecting the presence of a specific nucleotide sequence in a sample. Probes specifically hybridize to a target nucleotide sequence, or the complementary sequence thereof, and may be single- or double-stranded.

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

As used herein, the phrase “hybridization conditions” or “stringent hybridization conditions” refers to hybridization conditions which can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “hybridization” or “specific hybridization” refers to hybridization between two polynucleotides under stringent hybridization conditions.

The term “specifically hybridize,” as used herein, refers to the ability of a polynucleotide to bind detectably and specifically to a target nucleotide sequence. Polynucleotides, oligonucleotides and fragments thereof specifically hybridize to target nucleotide sequences under hybridization and wash conditions that minimize appreciable amounts of detectable binding to non-specific nucleic acids. High stringency conditions can be used to achieve specific hybridization conditions as is known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

As used herein, the term “hybridizes under stringent conditions” describes conditions for hybridization and washing under which nucleotide sequences having at least 30%, 35%, 40%, 45%, 50%, 55%, 60%, 65%, 70%, 75%, 80%, 85%, 90%, 95%, or more base pair matches to each other typically remain hybridized to each other.

The term “corresponding to” refers to a polynucleotide sequence that is identical to all or a portion of a reference polynucleotide sequence. In contradistinction, the term “complementary to” is used herein to indicate that a polynucleotide sequence is identical to all or a portion of the complementary strand of a reference polynucleotide sequence.

The terms “target sequence” or “target nucleotide sequence,” as used herein, refer to a particular nucleic acid sequence in a test sample to which a primer and/or probe is intended to specifically hybridize. A “target sequence” is typically longer than the primer or probe sequence and thus can contain multiple “primer target sequences” and “probe target sequences.” A target sequence may be single or double stranded. The term “primer target sequence” as used herein refers to a nucleic acid sequence in a test sample to which a primer is intended to specifically hybridize. The term “probe target sequence” refers to a nucleic acid sequence in a test sample to which a probe is intended to specifically hybridize.

As used herein an “amplified target polynucleotide sequence product” or “amplified product” or “amplification product” refers to the resulting amplicon from an amplification reaction such as a polymerase chain reaction. The resulting amplicon product arises from hybridization of complementary primers to a target polynucleotide sequence under suitable hybridization conditions and the repeating in a cyclic manner the polymerase chain reaction as catalyzed by DNA polymerase for DNA amplification or RNA polymerase for RNA amplification.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” or “amplified product” or “amplification product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through September 2005, John Wiley & Sons (hereinafter “Ausubel et al.”).

The term “end-point analysis” refers to a method where data collection occurs only when a reaction is substantially complete.

The term “real-time analysis” refers to periodic monitoring during PCR. Certain systems such as the Applied Biosystems 7500 Real-Time PCR System (Applied Biosystems, Foster City, Calif.) conduct monitoring during each thermal cycle at a pre-determined or user-defined point. Real-time analysis of PCR with FRET probes measures fluorescent dye signal changes from cycle-to-cycle, preferably minus any internal control signals.

As used herein, the term “sample” is a portion of a larger source. A sample is optionally a solid, gaseous, or fluidic. A sample is illustratively an environmental or biological sample. An environmental sample is illustratively, but not limited to water, sewage, soil, or air. A “biological sample” is as sample obtained from a biological organism, a tissue, cell, cell culture medium, or any medium suitable for mimicking biological conditions. Non-limiting examples include urine, saliva, gingival secretions, cerebrospinal fluid, gastrointestinal fluid, mucous, urogenital secretions, synovial fluid, blood, serum, plasma, feces, cystic fluid, lymph fluid, ascites, pleural effusion, interstitial fluid, intracellular fluid, ocular fluids, seminal fluid, mammary secretions, vitreal fluid, throat and nasal secretions, and the like. Methods of obtaining a sample are known in the art. In one embodiment of the present invention, the sample is urine and is collected by any method well known in the art including by catherization of a feline subject and, optionally, may be processed to obtain the final sample.

As used herein, the term “medium” refers to any liquid or fluid that may or may not contain one or more bacteria. A medium is illustratively a solid sample that has been suspended, solubilized, or otherwise combined with fluid to form a fluidic sample. Non-limiting examples include buffered saline solution, cell culture medium, acetonitrile, trifluoroacetic acid, combinations thereof, or any other fluid recognized in the art as suitable for combination with bacteria or other cells, or for dilution of a biological sample or amplification product for analysis.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “fibrotic disorder” or “fibrotic disease” refers to a medical condition featuring progressive and/or irreversible fibrosis, wherein excessive deposition of extracellular matrix occurs in and around inflamed or damaged tissue.

The polymerase chain reaction (PCR) is a primer extension reaction that provides a method to amplify a specific DNA or polynucleotide in vitro, generating thousands to millions of copies of a particular DNA sequence. PCR is now a common and often indispensable technique used in medical and biological research labs for a variety of applications. These include DNA cloning for sequencing, DNA-based phylogeny, or functional analysis of genes; the diagnosis of hereditary diseases; the identification of genetic fingerprints; and the detection and diagnosis of infectious diseases. Some of the variations of the basic PCR include quantitative real-time PCR (qPCR or RT-PCR), allele specific PCR, asymmetric PCR, hot start PCR, reverse transcription PCR, multiplex-PCR, nested-PCR, ligation-mediated PCR, Intersequence-specific PCR, Thermal asymmetric interlaced PCR and touchdown-PCR. These PCR variations provide wide variety of uses for different purposes. For example, single-nucleotide polymorphisms (SNPs) (single-base differences in DNA) can be identified by allele-specific PCR, qPCR can provide a very high degree of precision in determining the number of copies amplified in the PCR reactions (Bartlett et al., “A Short History of the Polymerase Chain Reaction”, PCR Protocols, 2003).

### II. CDK: Chronic Kidney Disease

One aspect of the present invention is directed to compositions and methods relating to an assay for the diagnosis and staging of Feline Chronic Kidney Disease (CDK). In one embodiment of the present invention, an assay that is rapid, reliable, and can be used for detecting the presence of a target indicator in body fluids, such as urine, is contemplated. It is especially advantageous that an assay in accordance with the present invention can not only be useful in diagnosing CDK, but also can distinguish early stage disease before clinical symptoms occur and during which intervention and treatment may limit progression of disease. In one embodiment, the assay used is a polymerase chain reaction (PCR), while another embodiment of the present invention uses reverse transcription PCR (RT-PCR) in an assay to diagnose and stage Feline CDK.

Yet another aspect of the present invention contemplates the use of some or all of the disclosed compositions useful in a CDK assay in a method to treat CKD, such as by preparing an antibody against on or more of the target antigens developed for the diagnostic assay and, subsequently, using the antibody in a composition to stimulate or enhance an immune response to renal disease in the subject by administering to the subject an amount of a disclosed composition to reduce or eliminate one or more symptoms of CKD in the subject.

CKD can start in cats as young as 5-6 years and prevalence has been reported to range from 30%-81% in geriatric cats, though purebreds may be more susceptible. The etiology of CKD is often idiopathic, though it can be secondary to acute insult. There are multiple potential predisposing factors with aging, stress, diet and periodontal disease most often indicated.

The pathology and etiology of CKD in cats is distinct from that in dogs and humans. In dogs and humans, CKD is marked by primary glomerulopathies and proteinuria while, in contrast the disease in cats is characterized as tubulointerstitial nephritis with progressive fibrosis, leading to significant cortical scarring and glomerulosclerosis.

### 1. Stages of CKD

CKD is categorized into disease stages established by the International Renal Interest Society (IRIS) based on serum creatinine measurements. Studies of the pathogenesis of CKD by stage show disease characteristics, as follows:

Stage I: The remaining normal renal parenchyma (51%-75%) is significantly less than in geriatric controls. Interstitial inflammation consisted exclusively of lymphocytes in a regionally extensive distribution, similar for all stages, but the severity of inflammation in stage I (<25%) was greater than in young cats and less than later CKD stages. Cortical and medullary scarring (the presence of collagenous matrix visible with trichrome stain) was nearly absent (<25% in a single cat) at this stage and significantly less than all other stages. Tubular degeneration was mild, focal to scattered, which was significantly less severe than later stages (stages III and IV). Single-cell necrosis of tubular epithelial cells—characterized by loss of basement membrane adhesion, pyknotic to karyorrhectic nuclei, and shrunken, hypereosinophilic cytoplasm—was infrequent and significantly less than that observed in geriatric controls and other CKD stages. Global glomerulosclerosis—in which >75% of the capillary tuft was effaced by extracellular matrix—was observed less frequently in stage I than all other CKD stages but was greater than that observed in young cats. Other glomi ernlar legions and vascular lesions were absent.

Stage II: The amount of remaining normal parenchyma (51%-75%) and severity of inflammation (<25%) were similar to that in stage I cats. Interstitial inflammation consisted of primarily lymphocytes and plasma cells and less frequently macrophages and granulocytes. Cortical and medullary scarring was significantly greater than in controls or CKD stage I cats but not different from stage III. Interstitial lipid was present in most cats and was more frequent than in controls. Mild to moderate tubular degeneration was observed and was greater than in both controls and less than that in stages III and IV. Epithelial single-cell necrosis was greater than in young cats or stage I cats but similar to geriatric control cats. Global glomerulosclerosis was significantly greater than in controls and stage I cats but significantly less than in later stages. Other glomerular lesions were identified in a few cats including membranoproliferative glomerulonephritis (MPGN) and focal segmental glomerulosclerosis (FSGS), cystic glomerular atrophy, or mesangial expansion. Bowman's capsule thickening with or without parietal cell hypertrophy was present in most cats. Vascular lesions included fibrointimal hyperplasia, hyperplastic arteriolosclerosis, hyalinosis, and torturous vessels in regions of scarring; their prevalence was not statistically different among groups.

Stage III: Significantly less normal parenchyma remained at this stage (25%-50%) compared with stages I and II. Interstitial inflammation (25%-50%) was greater than earlier CKD stages and less than in stage IV cats and appeared as regionally extensive infiltrates of lymphocytes accompanied in half of cases by plasma cells, macrophages, and granulocytes. Severity of renal scarring (<25%) was similar between stage II and stage III cats; cortical scarring was significantly less compared with stage IV. Tubular degeneration was moderate to severe and significantly greater than in controls and stages I and II. Global glomerulosclerosis (note that sclerosis is generally considered to be secondary to chronic fibrosis) was greater than in controls and earlier stages but less than in stage IV cats. Thickening of Bowman's capsule and parietal cell hypertrophy often present. Hyperplastic arteriolosclerosis was observed more frequently than in young cats, but was not different from other stages.

Stage IV: Significantly less normal parenchyma compared to other stages. Inflammation affecting 51% to 75% of the tissue section was significantly greater than in all other groups and consisted of lymphocytes and plasma cells in most cats. Cortical scarring (25%-50%) was typically greater than medullary scarring (<25%); cortical scarring was significantly greater in stage IV than all other groups, while medullary scarring was significantly different only from controls and stage I cats. Regionally extensive scarring was most frequently encountered and was significantly different from controls and stages I and II. Interstitial lipid was present in all cats. Tubular degeneration, affecting entire nephrons, and single-cell necrosis of tubular epithelial cells were significantly more severe than in controls and earlier stages but similar to stage III cats. Tubular dilation and cysts were more prevalent than in stage I or Stage II, respectively. Global glomerulosclerosis was the most severe at this stage compared with all other groups. Most cats had at least one other glomerular lesion, including FSGS, glomerular hypertrophy, mesangial expansion, endothelial hypertrophy, MPGN pattern, and cystic glomerular hypertrophy; these were significantly more prevalent than in controls and stage II cats. Kidneys frequently contained fibrointimal hyperplasia but infrequently were affected by hyperplastic arteriolosclerosis. The prevalence of vascular lesions was not significantly different from controls or other stages.

Normal parenchyma was unaffected by degeneration, atrophy, inflammation, or fibrosis was significantly less in later stages of CKD (ie, stages III and IV) compared with earlier stages (stages I and II) but similar between stages I and II. Interestingly, as little as 25% to 50% of the parenchyma was affected in the earlier stages of CKD, implying that even a mild degree of lesions could have functional significance. This is in contrast to the dogma that at least 75% of functional mass must be lost before clinical evidence of renal disease is evident.

Interstitial fibrosis and scarring, confirmed by Masson's trichrome stain, was statistically greater in stage IV compared with all other stages. Cats in stage IV were most likely to have 25% to 50% of their kidneys affected by scarring in comparison to <25% scarring in other stages. Interstitial fibrosis did not increase significantly between cats in stage II and III. This is in contrast to a previous study (which did not evaluate tissues stained with trichrome) in which interstitial fibrosis was the lesion that best correlated with severity of azotemia. This suggests that additional pathologic processes other than fibrosis are involved in disease progression and implies that initiation of any potential antifibrotic therapies in CKD cats should occur prior to stage IV, when irreversible fibrosis is most severe. This would be compatible with inflammation preceding and inducing fibrosis. However, patterns of scarring did not parallel that of interstitial inflammation, and a significant progression in scarring patterns from focal to regional to diffuse with increasing IRIS stage was not found. This suggests that instigators of fibrosis other than inflammation may be players in the progression of CKD and should be identified and evaluated as potential therapeutic targets.

A pathology summary from staging separate report shows that the severity of tubular degeneration, interstitial inflammation, fibrosis, and glomerulosclerosis was significantly greater in later stages of CKD compared with early stages of disease. Proteinuria was associated with increased severity of tubular degeneration, inflammation, fibrosis, tubular epithelial single-cell necrosis, and decreased normal parenchyma. Presence of hyperplastic arteriolosclerosis, fibrointimal hyperplasia, or other vascular lesions were not found to be significantly different between hypertensive and normotensive cats. The greater prevalence and severity of irreversible lesions in stage III and IV CKD implies that therapeutic interventions should be targeted at earlier stages of disease (McLeland, S. M. et al., Vet Pathol., 52(3):524-534 (2015)). Proteinuria, anemia, and hyperphosphatemia predict progression in feline CKD. These changes might reflect more progressive types of renal disease. Alternatively, they might reflect mechanisms of CKD progression such as tubular protein overload, hypoxia, and nephrocalcinosis (Chakrabarti, S. et al. J Vet Intern Med., 26:275-281 (2012)). Renal scarring encompasses interstitial fibrosis, which is an increase in extracellular matrix, as well as glomerulosclerosis and tubular atrophy. Tubulointerstitial changes, including fibrosis, are present in the early stages of feline CKD and become more severe in advanced disease. Collectively, these changes imply a loss of function and are considered, at least to date, irreversible. However, not all injury leads to irreversible damage. Replication and repair can lead to a return of normal function. Inflammation, edema, and tubular epithelial damage have the potential to resolve (ie, reversible) (McLeland, S. M. et al., Vet Pathol., 52(3):524-534 (2015)).

### 2. Etiology of CKD

The etiology of CKD is often unknown, but breed (purebreds at higher risk), age, and presence/severity of periodontal disease (1.5× more at risk than cats without periodontal disease) are major risk factors for the development of chronic kidney disease in domestic cats. The RVC retrospective study concluded that “These conditions are complex-meaning that there are many factors relating to genetics, lifestyle and environment that cumulatively determine whether an individual may develop either CKD or hypertension in their lifetime.”

In humans, the leading cause for end-stage renal failure is type 2 diabetes mellitus and hypertension. However, diabetic nephropathy has not been identified in cats, and renal lesions in diabetic cats have been no different from those in nondiabetic cats. In contrast to people and dogs, primary glomerulopathies with marked proteinuria are remarkably rare findings in cats. Although a variety of primary renal diseases have been implicated, the disease is idiopathic in most cats.

A variety of factors—including aging, ischemia, comorbid conditions, phosphorus overload, and routine vaccinations—have been implicated as factors that could contribute to the initiation of this disease in affected cats. Aging and stress seem to play an important role.

Other factors linked to renal disease include congenital malformation of the kidneys (birth defects), chronic bacterial infection of the kidneys with or without kidney stones (pyelonephritis), high blood pressure (hypertension), diseases associated with the immune system (glomerulonephritis, systemic lupus), and acute kidney disease (for example, poisoning with antifreeze that damages the kidneys can lead to chronic kidney disease).

Common clinical signs of CKD include drinking too much (polydipsia) and urinating large volumes of urine (polyuria), incontinence (leaking urine), especially at night, vomiting and/or diarrhea, lack of appetite and weight loss, general depression related to the elevation of waste products in the blood, anemia resulting in pale gums and weakness due to a low blood count, Gingivitis and overall weakness from low blood potassium. Less common signs of CKD include weakened bones can result in bone fractures, high blood pressure can lead to sudden blindness, itchy skin from calcium and phosphorous deposits and bleeding into the stomach or gut or bruising of skin. The most frequent morphologic diagnosis in cats with CKD is chronic tubulointerstitial nephritis and fibrosis, which are relatively nonspecific lesions.

### 3. Pathogenesis of CKD

Factors that are related to progression of established CKD, which occurs in some but not all cats, include dietary phosphorus intake, magnitude of proteinuria, and anemia. Renal fibrosis, a common histologic feature of aged feline kidneys, interferes with the normal relationship between peritubular capillaries and renal tubules. Experimentally, renal ischemia results in morphologic changes similar to those observed in spontaneous CKD. Renal hypoxia, perhaps episodic, may play a role in the initiation and progression of this disease (Brown, C. A. et al., Vet Pathol., 53(2):309-326 (2016)).

Factors that are related to progression of established CKD, which occurs in some but not all cats, include dietary phosphorus intake, magnitude of proteinuria, and anemia. Renal fibrosis, a common histologic feature of aged feline kidneys, interferes with the normal relationship between peritubular capillaries and renal tubules. Experimentally, renal ischemia results in morphologic changes similar to those observed in spontaneous CKD. Renal hypoxia, perhaps episodic, may play a role in the initiation and progression of this disease.

A recent publication comparing renal disease in wild and captive cheetahs showed that “renal medullary fibrosis was the only lesion associated with the likelihood of death being due to chronic renal disease, and cheetahs with this lesion were younger, on average, than cheetahs with other renal lesions” (Mitchell E. P, PLoS ONE, 13(3):e0194114 (2018)). These results suggest that age and renal medullary fibrosis are the primary factors influencing the pathogenesis of chronic renal disease in captive cheetahs. Apart from amyloidosis, these findings are analogous to those described in chronic renal disease in domestic cats, which is postulated to result primarily from repetitive hypoxic injury of renal tubules, mediated by age and stress.”

### 4. CKD and Fibrosis

Progressive scarring (fibrosis) is a pathological feature of many chronic inflammatory diseases, and is an important cause of morbidity and mortality worldwide. Fibrosis is characterized by the accumulation of excess extracellular matrix components (e.g., collagen, fibronectin) that forms fibrous connective tissue in and around an inflamed or damaged tissue. Fibrosis may cause overgrowth, hardening, and/or scarring that disrupts the architecture of the underlying organ or tissue. While controlled tissue remodeling and scarring is part of the normal wound healing process promoted by transdifferentiation of fibroblasts into myofibroblasts, excessive and persistent scarring due to severe or repetitive injury or dysregulated wound healing (e.g., persistence of myofibroblasts) can eventually result in permanent scarring, organ dysfunction and failure, and even death.

Fibrotic changes can occur in vascular disorders (e.g., peripheral vascular disease, cardiac disease, cerebral disease) and in all main tissue and organ systems (e.g., lung, liver, kidney, heart, skin). Fibrotic disorders include a wide range of clinical presentations, including multisystemic disorders, such as systemic sclerosis, multifocal fibrosclerosis, and organ-specific disorders, such as pulmonary, liver, and kidney fibrosis (Rosenbloom et al., Ann. Intern. Med. 152:159, 2010; Wynn, Nat. Rev. Immunol. 4:583, 2004). While the etiology and causative mechanisms of individual fibrotic disorders may vary (e.g., ischemic event, exposure to a chemical, radiation, or infectious agent) and are poorly understood, they all share the common feature of abnormal and excessive deposition of extracellular matrix in affected tissues (Wynn and Ramalingam, Nat. Med. 18:1028, 2012).

In certain embodiments, a fibrotic disorder or disease is associated with the persistent presence of myofibroblasts in and around fibrotic foci or lesions. Excessive and persistent fibrosis can progressively remodel and destroy normal tissue, which may lead to dysfunction and failure of affected organs, and ultimately death. A fibrotic disorder may affect any tissue in the body and is generally initiated by an injury and the transdifferentiation of fibroblasts into myofibroblasts. As used herein, “transdifferentiation” refers to the direct conversion of one cell type into another. It is to be understood that fibrosis alone triggered by normal wound healing processes that has not progressed to a pathogenic state is not considered a fibrotic disorder or disease of this disclosure. A “fibrotic lesion” or “fibrotic plaque” refers to a focal area of fibrosis.

Non-limiting examples of fibrotic disorders or fibrotic diseases include pulmonary fibrosis, idiopathic pulmonary fibrosis, cystic fibrosis, liver fibrosis (e.g., cirrhosis), cardiac fibrosis, endomyocardial fibrosis, vascular fibrosis (e.g., atherosclerosis, stenosis, restenosis), atrial fibrosis, mediastinal fibrosis, myelofibrosis, retroperitoneal fibrosis, progressive massive fibrosis (e.g., lungs), chronic kidney disease, nephrogenic systemic fibrosis, Crohn's disease, hypertrophic scarring, keloid, scleroderma, systemic sclerosis (e.g., skin, lungs), athrofibrosis (e.g., knee, shoulder, other joints), Peyronie's disease, Dupuytren's contracture, adhesive capsulitis, organ transplant associated fibrosis, ischemia associated fibrosis, or the like. Myofibroblasts are the principal cells responsible for extracellular matrix (ECM) production. Generally, myofibroblasts do not exist in normal kidneys, while 50% of MFs in fibrotic kidneys are derive from renal resident fibroblasts (LeBleu et al., 2013). The activation of fibroblasts and excessive deposition of the ECM leads to damage of the renal parenchyma and progressive loss of renal function, which eventually progresses to end-stage renal disease (ESRD). Thus, renal fibrosis is the hallmark of CKD, and it has been demonstrated that the activation of fibroblasts leads to fibrosis (Grgic et al., 2012).

### III. Compositions for Treating Feline Chronic Kidney Disease (CKD)

Compositions and methods thereof for treating feline CKD as well as for diagnosing and staging disease are provided herein. In some embodiments, the disclosed compositions and methods are useful for limiting progression of renal disease.

### 1. CAR T Cells

Chimeric antigen receptors (CARs, also known as chimeric T cell receptors) are synthetic constructs that are designed to be expressed in host T cells or NK cells and to induce an immune response against a specific target antigen and cells expressing that antigen. The term “chimeric receptor” as used herein is defined as a cell-surface receptor comprising an extracellular ligand binding domain, a transmembrane domain and a cytoplasmic co-stimulatory signaling domain in a combination that is not naturally found together on a single protein. This particularly includes receptors wherein the extracellular domain and the cytoplasmic domain are not naturally found together on a single receptor protein. Further, the chimeric receptor is different from the TCR expressed in the native T cell lymphocyte.

As described in U.S. Pat. Nos. 5,359,046, 5,686,281 and 6,103,521, the extracellular domain may be obtained from any of the wide variety of extracellular domains or secreted proteins associated with ligand binding and/or signal transduction. The extracellular domain may be part of a protein which is monomeric, homodimeric, heterodimeric, or associated with a larger number of proteins in a non-covalent complex. In particular, the extracellular domain may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used or a truncated chain may be used, where all or a part of the CH1, CH2, or CH3 domains may be removed or all or part of the hinge region may be removed.

As described herein, the extracellular domains of CARs are often derived from immunoglobulins. The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Numerous publications discuss the use of phage display technology to produce and screen libraries of polypeptides for binding to a selected analyte. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods, a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present. The antibodies so identified may then be further analyzed for affinity and specificity in the CAR design selected.

The transmembrane domain may be contributed by the protein contributing the multispecific extracellular inducer clustering domain, the protein contributing the effector function signaling domain, the protein contributing the proliferation signaling portion, or by a totally different protein. For the most part it will be convenient to have the transmembrane domain naturally associated with one of the domains. In some cases, it will be desirable to employ the transmembrane domain of the C, 11 or FcERly chains which contain a cysteine residue capable of disulfide bonding, so that the resulting chimeric protein will be able to form disulfide linked dimers with itself, or with unmodified versions of the C, 11 or FcERly chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of C, q or FcERly chains and −13, MB1 (Iga), B29 or CD3y, C, or E, in order to retain physical association with other members of the receptor complex. Examples of suitable transmembrane regions for use with the invention include the constant (Fc) regions of immunoglobins, human CD8a, and artificial linkers that serve to move the targeting moiety away from the cell surface for improved access to and binding on target cells, however any transmembrane region sufficient to anchor the CAR in the membrane can be used. Persons of skill are aware of numerous transmembrane regions and the structural elements (such as lipophilic amino acid regions) that produce transmembrane domains in numerous membrane proteins and therefore can substitute any convenient sequence.

The cytoplasmic domain of the chimeric receptors of the invention can comprise a signaling domain (e.g., co-stimulatory signaling domain) by itself or combined with any other desired cytoplasmic domain(s) useful in the context of this chimeric receptor type, such as for example, a 4-1BB signaling domain, a CD3C signaling domain and/or a CD28 signaling domain. The 4-1BB, CD3C and CD28 signaling domains are well characterized, including for example, their use in chimeric receptors.

In adoptive immunotherapy, the patient's circulating lymphocytes, or tumor infiltrated lymphocytes, are isolated in vitro, activated by lymphokines such as IL-2 or transduced with genes for tumor necrosis, and readministered. To achieve this, one would administer to an animal, or human patient, an immunologically effective amount of activated lymphocytes genetically modified to express a tumor-specific chimeric receptor gene as described herein. The activated lymphocytes will most preferably be the patient's own cells that were earlier isolated from a blood or tumor sample and activated and expanded in vitro. The antigen-specific CAR-T cells can be expanded in vitro for use in adoptive cellular immunotherapy in which infusions of such cells have been shown to have anti-tumor reactivity in a tumor-bearing host.

Genetic modification for introduction of the CAR construct into T cells can be accomplished by transducing (or otherwise delivering) a T cell composition with a recombinant DNA or RNA construct, such as for example, a vector. A vector may be any agent capable of delivering or maintaining nucleic acid in a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

Selection of promoter and other regulatory sequences for protein expression are well known to those of skill in the art. Cell specific promoters for expression in T-cells include, but are not limited to, human CD2, distal Lck, and proximal Lck. In other embodiments, non-tissue specific promoters such as non-tissue specific promoters including viral promoters such as cytomegalovirus (CMV) promoter, I3-actin promoter phosphoglycerate kinase (PGK) promoter, ubiquitin promoter, and EF-1a promoter can be used. This list is not meant to be limiting. An expression construction preferably also includes sequences to allow for the replication of the expression construct. Transcription of the DNA encoding the polypeptides of the present invention by higher eukaryotes may be increased by inserting an enhancer sequence into the vector. Enhancers are cis-acting elements of DNA, usually about from 10 to 300 by that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers. Preferably, a retroviral vector (either gamma-retroviral or lentiviral) is employed for the introduction of the CAR nucleic acid construct into the cell. For example, a polynucleotide encoding a co-stimulatory ligand protein (e.g., tumor necrosis factor (TNF) ligand, such as 4-1BBL, OX4OL, CD70, LIGHT, and CD3OL, or an Ig superfamily ligand, such as CD80 and CD86), or a receptor that binds an antigen, or a variant, or a fragment thereof, can be cloned into a retroviral vector and expression can be driven from its endogenous promoter, from the retroviral long terminal repeat, or from a promoter specific for a target cell type of interest. Non-viral vectors may be used as well.

### 2. CAR NK Cells

CAR constructs have been used to direct natural killer (NK) cell activity, as reviewed by Hermanson & Kaufman (2015, Front Immunol 6:195) and Carlsten & Childs (2015, Front Immunol 6:266). Like T cells, NK cells can be transfected with CAR expression constructs and used to induce an immune response. Because NK cells do not require HLA matching, they can be used as allogeneic effector cells (Harmanson & Kaufman, 2015). Also, peripheral blood NK cells (PB-NK), of use for therapy, may be isolated from donors by a simple blood draw. The CAR constructs of use may contain similar elements to those used to make CAR-T cells. CAR-NK cells may contain a targeting molecule, such as a scFV or Fab, that binds to a disease associated antigen, such as a tumor-associated antigen (TAA), or to a hapten on a targetable construct. This avoids the problem that NK cells, unlike T cells, lack antigen specificity for targeting cells to be killed. The cell-targeting scFv or Fab may be linked via a transmembrane domain to one or more intracellular signaling domains to effect lymphocyte activation. Signaling domains used with CAR-NK cells have included CD3-ζ, CD28, 4-1BB, DAP10 and OX40. NK cell lines of use have included NK-92, NKG, YT, NK-YS, HANK-1, YTS and NKL cells. Transfection with genes encoding IL-2 and/or IL-15 has been proposed to reduce dependence on the need for exogenous cytokines for in vivo persistence and cell population expansion. Clinical trials using NK cells from haploidentical donors have demonstrated long-term remissions in patients with refractory acute myelogenous leukemia (Miller et al., 2004, Blood 105:3051-57). Efficacy has also been demonstrated against breast and ovarian cancer (Geller et al., 2011, Cytotherapy 13:98-107).

Nucleotide sequences encoding the cDNA of CAR constructs are incorporated in an expression vector, such as a retroviral or lentiviral vector, for transfer into T cells or NK cells. Following infection, transfection, lipofection or alternative means of introducing the vector into the host cell (CAR-T or CAR-NK), the cells are administered to a subject to induce an immune response against antigen-expressing target cells. Binding of CARs on the surface of transduced T cells or NK cells to antigens expressed by a target cells activates the T or NK cell. Activation of T or NK cells by CARs does not require antigen processing and presentation by the HLA system. A major concern with CAR-T therapy is the danger of a “cytokine storm” associated with intense antitumor responses mediated by large numbers of activated T cells (Sadelain et al., Cancer Discov 3:388-98, 2013). Side effects can include high fever, hypotension and/or organ failure, potentially resulting in death. The cytokines produced by CAR-NK cells differ from CAR-T cells, reducing the risk of an adverse cytokine-mediated reaction.

### 3. T Cell Receptors

T cell receptors (TCR) are cell surface receptors that participate in the activation of T cells in response to the presentation of antigen. The TCR is generally made from two chains, alpha and beta, which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains is generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of GVHD. It has been shown that normal surface expression of the TCR depends on the coordinated synthesis and assembly of all seven components of the complex (Ashwell and Klusner 1990). The inactivation of TCRalpha or TCRbeta can result in the elimination of the TCR from the surface of T cells preventing recognition of alloantigen and thus GVHD. However, TCR disruption results in the elimination of the CD3 signaling component and alters the means of further T cell expansion.

### 4. Fibroblast Activation Protein (FAP)

Human Fibroblast Activation Protein (FAP; GenBank Accession Number AAC51668; NCBI Reference Sequence: NM 004460.3), also known as Seprase, is a 170 kDa integral membrane serine peptidase (EC 3.4.21.B28). Together with dipeptidyl peptidase IV (DPPIV, also known as CD26; GenBank Accession Number P27487), a closely related cell-surface enzyme, and other peptidases, FAP belongs to the dipeptidyl peptidase IV family (Yu et al., FEBS J. 277 (2010), 1126-1144). It is a homodimer containing two N-glycosylated subunits with a large C-terminal extracellular domain, in which the enzyme's catalytic domain is located (Scanlan et al., Proc. Natl. Acad. Sci. USA 91 (1994), 5657-5661). FAP, in its glycosylated form, has both post-prolyl dipeptidyl peptidase and gelatinase activities (Sun et al., Protein Expr. Purif. 24 (2002), 274-281). Thus, FAP is a serine protease with both dipeptidyl peptidase, as well as endopeptidase activity cleaving gelatin and type I collagen.

Human FAP was originally identified in cultured fibroblasts using the monoclonal antibody (mAb) F19 (described in WO 93/05804, ATCC Number HB 8269). Homologues of the protein were found in several species, including mice (Niedermeyer et al., Int. J. Cancer 71, 383-389 (1997), Niedermeyer et al., Eur. J. Biochem. 254, 650-654 (1998); GenBank Accession Number AAH19190; NCBI Reference Sequence: NP_032012.1). Human and murine FAP share an 89% sequence identity and have similar functional homology. FAP has a unique tissue distribution: its expression was found to be highly upregulated on reactive stromal fibroblasts of more than 90% of all primary and metastatic epithelial tumors, including lung, colorectal, bladder, ovarian and breast carcinomas, while it is generally absent from normal adult tissues (Rettig et al., Proc. Natl. Acad. Sci. USA 85 (1988), 3110-3114; Garin-Chesa et al., Proc. Natl. Acad. Sci. USA 87 (1990), 7235-7239).

### 5. Immunoliposomes

Liposomes are spherical vesicles comprised of concentrically ordered lipid bilayers that encapsulate an aqueous phase. Liposomes serve as a delivery vehicle for therapeutic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, so-called Stealth® or long-circulating liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Targeted liposomes have targeting ligands or affinity moieties attached to the surface of the liposomes. The targeting ligands may be antibodies or fragments thereof, in which case the liposomes are referred to as immunoliposomes. When administered systemically targeted liposomes deliver the entrapped therapeutic agent to a target tissue, region or, cell. Because targeted liposomes are directed to a specific region or cell, healthy tissue is not exposed to the therapeutic agent. Such targeting ligands can be attached directly to the liposomes' surfaces by covalent coupling of the targeting ligand to the polar head group residues of liposomal lipid components (see, for example, U.S. Pat. No. 5,013,556). This approach, however, is suitable primarily for liposomes that lack surface-bound polymer chains, as the polymer chains interfere with interaction between the targeting ligand and its intended target (Klibanov, A. L., et al., Biochim. Biophys. Acta., 1062:142-148 (1991); Hansen, C. B., et al., Biochim. Biophys. Acta, 1239:133-144 (1995)).

Alternatively, the targeting ligands can be attached to the free ends of the polymer chains forming the surface coat on the liposomes (Allen. T. M., et al., Biochim. Biophys. Acta, 1237:99-108 (1995); Blume, G. et al., Biochim. Biophys. Acta, 1149:180-184 (1993)). In this approach, the targeting ligand is exposed and readily available for interaction with the intended target.

Liposomes suitable for use in the composition of the present invention include those composed primarily of vesicle-forming lipids. Such a vesicle-forming lipid is one which can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes.

The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose carbon chains have varying degrees of saturation can be obtained commercially or prepared according to published methods. Other suitable lipids include glycolipids, cerebrosides and sterols, such as cholesterol.

Cationic lipids are also suitable for use in the liposomes of the invention, where the cationic lipid can be included as a minor component of the lipid composition or as a major or sole component. Such cationic lipids typically have a lipophilic moiety, such as a sterol, an acyl or diacyl chain, and where the lipid has an overall net positive charge. Preferably, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3[N-(N′,N′-dimethylaminoethane) carbamoly]cholesterol (DC-Chol); and dimethyldioctadecylammonium (DDAB). The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

The vesicle-forming lipid can be selected to achieve a specified degree of fluidity or rigidity, to control the stability of the liposome in serum, to control the conditions effective for insertion of the targeting conjugate, as will be described, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

On the other hand, lipid fluidity is achieved by incorporation of a relatively fluid lipid, typically one having a lipid phase with a relatively low liquid to liquid-crystalline phase transition temperature, e.g., at or below room temperature.

The liposomes may also include a vesicle-forming lipid covalently attached to a hydrophilic polymer, also referred to herein as a “lipopolymer”. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating.

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

Preparation of vesicle-forming lipids derivatized with hydrophilic polymers has been described, for example in U.S. Pat. No. 5,395,619. Preparation of liposomes including such derivatized lipids has also been described, where typically between 1-20 mole percent of such a derivatized lipid is included in the liposome formulation (see, for example, U.S. Pat. No. 5,013,556).

### 6. Liposome Preparation

Various approaches have been described for preparing liposomes having a targeting ligand attached to the distal end of liposome-attached polymer chains. One approach involves preparation of lipid vesicles which include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, for example, U.S. Pat. Nos. 6,224,903, 5,620,689). In yet another approach, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973, 6,316,024).

Liposomes carrying an entrapped agent and bearing surface-bound targeting ligands, i.e., targeted, therapeutic liposomes, are prepared by any of these approaches. A preferred method of preparation is the insertion method, where pre-formed liposomes and are incubated with the targeting conjugate to achieve insertion of the targeting conjugate into the liposomal bilayers. In this approach, liposomes are prepared by a variety of techniques, such as those detailed in Szoka, F., Jr., et al., Ann. Rev. Biophys. Bioeng., 9:467 (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

The liposomes can include a vesicle-forming lipid derivatized with a hydrophilic polymer to form a surface coating of hydrophilic polymer chains on the liposomes surface. Addition of a lipid-polymer conjugate is optional, since after the insertion step, described below, the liposomes will include lipid-polymer-targeting ligand. Additional polymer chains added to the lipid mixture at the time of liposome formation and in the form of a lipid-polymer conjugate result in polymer chains extending from both the inner and outer surfaces of the liposomal lipid bilayers. Addition of a lipid-polymer conjugate at the time of liposome formation is typically achieved by including between 1-20 mole percent of the polymer-derivatized lipid with the remaining liposome forming components, e.g., vesicle-forming lipids. Exemplary methods of preparing polymer-derivatized lipids and of forming polymer-coated liposomes have been described in U.S. Pat. Nos. 5,013,556, 5,631,018 and 5,395,619, which are incorporated herein by reference. It will be appreciated that the hydrophilic polymer may be stably coupled to the lipid, or coupled through an unstable linkage, which allows the coated liposomes to shed the coating of polymer chains as they circulate in the bloodstream or in response to a stimulus.

The liposomes also include a therapeutic or diagnostic agent, and exemplary agents are provided below. The selected agent is incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse-phase evaporation, are also suitable.

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS—MANUFACTURING AND PRODUCTION TECHNOLOGY, P. Tyle, Ed., Marcel Dekker, New York, pp. 267-316 (1990)).

After formation of the liposomes, a targeting ligand is incorporated to achieve a cell-targeted, therapeutic liposome. The targeting ligand is incorporated by incubating the pre-formed liposomes with the lipid-polymer-ligand conjugate, prepared as described above. The pre-formed liposomes and the conjugate are incubated under conditions effective to association with the conjugate and the liposomes, which may include interaction of the conjugate with the outer liposome bilayer or insertion of the conjugate into the liposome bilayer. More specifically, the two components are incubated together under conditions which achieve associate of the conjugate with the liposomes in such a way that the targeting ligand is oriented outwardly from the liposome surface, and therefore available for interaction with its cognate receptor. It will be appreciated that the conditions effective to achieve such association or insertion are determined based on several variables, including, the desired rate of insertion, where a higher incubation temperature may achieve a faster rate of insertion, the temperature to which the ligand can be safely heated without affecting its activity, and to a lesser degree the phase transition temperature of the lipids and the lipid composition. It will also be appreciated that insertion can be varied by the presence of solvents, such as amphipathic solvents including polyethyleneglycol and ethanol, or detergents.

The targeting conjugate, in the form of a lipid-polymer-ligand conjugate, will typically form a solution of micelles when the conjugate is mixed with an aqueous solvent. The micellar solution of the conjugates is mixed with a suspension of pre-formed liposomes for incubation and association of the conjugate with the liposomes or insertion of the conjugate into the liposomal lipid bilayers. The incubation is effective to achieve associate or insertion of the lipid-polymer-antibody conjugate with the outer bilayer leaflet of the liposomes, to form an immunoliposome.

After preparation, the immunoliposomes preferably have a size of less than about 150 nm, preferably of between about 85-120 nm, and more preferably of between 90-110 nm, as measured, for example, by dynamic light scattering at 30° or 90°.

7. Lipid nanoparticles (LNP) for mRNA Delivery

Conventional gene therapy involves the use of DNA for insertion of desired genetic information into host cells. The DNA introduced into the cell is usually integrated to a certain extent into the genome of one or more transfected cells, allowing for long-lasting action of the introduced genetic material in the host. While there may be substantial benefits to such sustained action, integration of exogenous DNA into a host genome may also have many deleterious effects. For example, it is possible that the introduced DNA will be inserted into an intact gene, resulting in a mutation which impedes or even totally eliminates the function of the endogenous gene. Thus, gene therapy with DNA may result in the impairment of a vital genetic function in the treated host, such as e.g., elimination or deleteriously reduced production of an essential enzyme or interruption of a gene critical for the regulation of cell growth, resulting in unregulated or cancerous cell proliferation. In addition, with conventional DNA based gene therapy it is necessary for effective expression of the desired gene product to include a strong promoter sequence, which again may lead to undesirable changes in the regulation of normal gene expression in the cell. It is also possible that the DNA based genetic material will result in the induction of undesired anti-DNA antibodies, which in turn, may trigger a possibly fatal immune response. Gene therapy approaches using viral vectors can also result in an adverse immune response. In some circumstances, the viral vector may even integrate into the host genome. In addition, production of clinical grade viral vectors is also expensive and time consuming. Targeting delivery of the introduced genetic material using viral vectors can also be difficult to control. Thus, while DNA based gene therapy has been evaluated for delivery of secreted proteins using viral vectors (U.S. Pat. No. 6,066,626; US2004/0110709; Amalfitano, A., et al, PNAS (1999) vol. 96, pp. 8861-66), these approaches may be limited for these various reasons.

The invention also provides compositions and methods for intracellular delivery of mRNA in a liposomal transfer vehicle to one or more target cells for production of therapeutic levels of protein. The term “functional,” as used herein to qualify a protein or enzyme, means that the protein or enzyme has biological activity, or alternatively is able to perform the same, or a similar function as the native or normally-functioning protein or enzyme. The mRNA compositions of the invention are useful for the treatment of a various metabolic or genetic disorders, and in particular those genetic or metabolic disorders which involve the non-expression, mis-expression or deficiency of a protein or enzyme. The term “therapeutic levels” refers to levels of protein detected in the blood or tissues that are above control levels, wherein the control may be normal physiological levels, or the levels in the subject prior to administration of the mRNA composition. The term “secreted” refers to protein that is detected outside the target cell, in extracellular space. The protein may be detected in the blood or in tissues. In the context of the present invention the term “produced” is used in its broadest sense to refer the translation of at least one mRNA into a protein or enzyme. As provided herein, the compositions include a transfer vehicle. As used herein, the term “transfer vehicle” includes any of the standard pharmaceutical carriers, diluents, excipients and the like which are generally intended for use in connection with the administration of biologically active agents, including nucleic acids. The compositions and in particular the transfer vehicles described herein are capable of delivering mRNA to the target cell. In embodiments, the transfer vehicle is a lipid nanoparticle. mRNA

The mRNA in the compositions of the invention may encode, for example, an encoded hormone, enzyme, receptor, polypeptide, peptide or other protein of interest may be one that is normally secreted or excreted. In alternate embodiments, the mRNA is engineered to encode a protein that is not normally secreted or excreted, operably linked to a signal sequence that will allow the protein to be secreted when it is expressed in the cells. In some embodiments of the invention, the mRNA may optionally have chemical or biological modifications which, for example, improve the stability and/or half-life of such mRNA or which improve or otherwise facilitate protein production. The methods of the invention provide for optional co-delivery of one or more unique mRNA to target cells, for example, by combining two unique mRNAs into a single transfer vehicle. In one embodiment of the present invention, a therapeutic first mRNA, and a therapeutic second mRNA, may be formulated in a single transfer vehicle and administered. The present invention also contemplates co-delivery and/or co-administration of a therapeutic first mRNA and a second nucleic acid to facilitate and/or enhance the function or delivery of the therapeutic first mRNA. For example, such a second nucleic acid (e.g., exogenous or synthetic mRNA) may encode a membrane transporter protein that upon expression (e.g., translation of the exogenous or synthetic mRNA) facilitates the delivery or enhances the biological activity of the first mRNA. Alternatively, the therapeutic first mRNA may be administered with a second nucleic acid that functions as a “chaperone” for example, to direct the folding of either the therapeutic first mRNA.

The methods of the invention also provide for the delivery of one or more therapeutic nucleic acids to treat a single disorder or deficiency, wherein each such therapeutic nucleic acid functions by a different mechanism of action. For example, the compositions of the present invention may comprise a therapeutic first mRNA which, for example, is administered to correct an endogenous protein or enzyme deficiency, and which is accompanied by a second nucleic acid, which is administered to deactivate or “knock-down” a malfunctioning endogenous nucleic acid and its protein or enzyme product. Such “second” nucleic acids may encode, for example mRNA or siRNA.

Upon transfection, a natural mRNA in the compositions of the invention may decay with a half-life of between 30 minutes and several days. The mRNA in the compositions of the invention preferably retain at least some ability to be translated, thereby producing a functional protein or enzyme. Accordingly, the invention provides compositions comprising and methods of administering a stabilized mRNA. In some embodiments of the invention, the activity of the mRNA is prolonged over an extended period of time. For example, the activity of the mRNA may be prolonged such that the compositions of the present invention are administered to a subject on a semi-weekly or bi-weekly basis, or more preferably on a monthly, bi-monthly, quarterly or an annual basis. The extended or prolonged activity of the mRNA of the present invention, is directly related to the quantity of protein or enzyme produced from such mRNA. Similarly, the activity of the compositions of the present invention may be further extended or prolonged by modifications made to improve or enhance translation of the mRNA. Furthermore, the quantity of functional protein or enzyme produced by the target cell is a function of the quantity of mRNA delivered to the target cells and the stability of such mRNA. To the extent that the stability of the mRNA of the present invention may be improved or enhanced, the half-life, the activity of the produced protein or enzyme and the dosing frequency of the composition may be further extended.

Accordingly, in some embodiments, the mRNA in the compositions of the invention comprise at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. The stabilized mRNA molecules provided herein demonstrate longer half-lives relative to their naturally occurring, unmodified counterparts (e.g. the wild-type version of the mRNA). Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozak consensus sequence). (Kozak, M., Nucleic Acids Res 15(20):8125-48 (1987)).

In some embodiments, the mRNA of the invention has undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid.

Contemplated modifications to the mRNA nucleic acids of the present invention also include the incorporation of pseudouridines. The incorporation of pseudouridines into the mRNA nucleic acids of the present invention may enhance stability and translational capacity, as well as diminishing immunogenicity in vivo. See, e.g., Kariko, K., et al, Molecular Therapy 16 (11):1833-1840 (2008). Substitutions and modifications to the mRNA of the present invention may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible). For example, the codons for Gly can be altered to GGA or GGG instead of GGU or GGC.

The term modification also includes, for example, the incorporation of non-nucleotide linkages or modified nucleotides into the mRNA sequences of the present invention (e.g., modifications to one or both the 3′ and 5′ ends of an mRNA molecule encoding a functional protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, in one embodiment a long poly A tail can be added to an mRNA molecule thus rendering the mRNA more stable. Poly A tails can be added using a variety of art-recognized techniques. For example, long poly A tails can be added to synthetic or in vitro transcribed mRNA using poly A polymerase (Yokoe, et al. Nature Biotechnology. 1996; 14:1252-1256). A transcription vector can also encode long poly A tails. In addition, poly A tails can be added by transcription directly from PCR products. In one embodiment, the length of the poly A tail is at least about 90, 200, 300, 400 at least 500 nucleotides. In one embodiment, the length of the poly A tail is adjusted to control the stability of a modified mRNA molecule of the invention and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In one embodiment, the stabilized mRNA molecules are sufficiently resistant to in vivo degradation (e.g., by nucleases), such that they may be delivered to the target cell without a transfer vehicle.

In one embodiment, an mRNA can be modified by the incorporation 3′ and/or 5′ untranslated (UTR) sequences which are not naturally found in the wild-type mRNA. In one embodiment, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

In some embodiments, the mRNA in the compositions of the invention include modification of the 5′ end of the mRNA to include a partial sequence of a CMV immediate-early 1 (IE1) gene, or a fragment thereof to improve the nuclease resistance and/or improve the half-life of the mRNA. In addition to increasing the stability of the mRNA nucleic acid sequence, it has been surprisingly discovered the inclusion of a partial sequence of a CMV immediate-early 1 (IE1) gene enhances the translation of the mRNA and the expression of the functional protein or enzyme. Also contemplated is the inclusion of a human growth hormone (hGH) gene sequence, or a fragment thereof to the 3′ ends of the nucleic acid (e.g., mRNA) to further stabilize the mRNA. Generally, preferred modifications improve the stability and/or pharmacokinetic properties (e.g., half-life) of the mRNA relative to their unmodified counterparts, and include, for example modifications made to improve such mRNA's resistance to in vivo nuclease digestion.

In some embodiments, the composition can comprise a stabilizing reagent. The compositions can include one or more formulation reagents that bind directly or indirectly to, and stabilize the mRNA, thereby enhancing residence time in the target cell. Such reagents preferably lead to an improved half-life of the mRNA in the target cells. For example, the stability of an mRNA and efficiency of translation may be increased by the incorporation of “stabilizing reagents” that form complexes with the mRNA that naturally occur within a cell (see e.g., U.S. Pat. No. 5,677,124). Incorporation of a stabilizing reagent can be accomplished for example, by combining the poly A and a protein with the mRNA to be stabilized in vitro before loading or encapsulating the mRNA within a transfer vehicle. Exemplary stabilizing reagents include one or more proteins, peptides, aptamers, translational accessory protein, mRNA binding proteins, carbohydrates, and/or translation initiation factors.

Stabilization of the compositions may also be improved by the use of opsonization-inhibiting moieties, which are typically large hydrophilic polymers that are chemically or physically bound to the transfer vehicle (e.g., by the intercalation of a lipid-soluble anchor into the membrane itself, or by binding directly to active groups of membrane lipids). These opsonization-inhibiting hydrophilic polymers form a protective surface layer which significantly decreases the uptake of the liposomes by the macrophage-monocyte system and reticulo-endothelial system (e.g., as described in U.S. Pat. No. 4,920,016, the entire disclosure of which is herein incorporated by reference). Transfer vehicles modified with opsonization-inhibition moieties thus remain in the circulation much longer than their unmodified counterparts.

### 8. Pharmaceutical Compositions

Pharmaceutical compositions including the disclosed immunoliposome agents are provided. Pharmaceutical compositions containing the immunomodulatory agent can be for administration by parenteral (intramuscular, intraperitoneal, intravenous (IV) or subcutaneous injection) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed immunomodulatory agents, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed immunomodulatory agents, generally dosage levels of 0.001 to 20 mg/kg of body weight daily are administered to mammals. Generally, for intravenous injection or infusion, dosage may be lower.

In certain embodiments, the immunomodulatory agent is administered locally, for example by injection directly into a site to be treated. Typically, the injection causes an increased localized concentration of the immunomodulatory agent composition which is greater than that which can be achieved by systemic administration. The immunomodulatory agent compositions can be combined with a matrix as described above to assist in creating an increased localized concentration of the polypeptide compositions by reducing the passive diffusion of the polypeptides out of the site to be treated.

### 9. Formulations for Parenteral Administration

In some embodiments, the disclosed antibody and fusion protein compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 10. Controlled Delivery Polymeric Matrices

The antibody and fusion protein compositions disclosed herein can also be administered in controlled release formulations. Controlled release polymeric devices can be made for long term release systemically following implantation of a polymeric device (rod, cylinder, film, disk) or injection (microparticles or nanoparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of fusion polypeptides or nucleic acids encoding the fusion polypeptides, although in some embodiments biodegradable matrices are preferred. These may be natural or synthetic polymers, although synthetic polymers are preferred in some embodiments due to the better characterization of degradation and release profiles. The polymer is selected based on the period over which release is desired. In some cases, linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### IV. Methods of Use for Immune Modulation

The disclosed compositions thereof can be used to modulate an immune response in a subject in need thereof. One embodiment provides a method of expressing CAR to induce T cell signaling in a subject in need thereof.

Methods of inducing or enhancing an immune response in a subject are provided. Typically, the methods include administering a subject an effective amount of one or more of the disclosed compositions thereof to immunospecifically reduce or block the immune suppressive signal of CAR, thus promoting an immune response. The immune response can be, for example inducing, promoting or enhancing T cell activation, secretion of cytokines by immune cells, T cell proliferation. The disclosed antibodies or antigen binding fragments thereof can be administered to a subject in need thereof in an effective amount to overcome T cell suppression. Overcoming T cell suppression can be determined by measuring T cell function using known techniques.

The methods can be used in vivo or ex vivo to induce, promote, or enhance a stimulating immune response.

In some embodiments, the antibody or antigen binding fragment thereof, or nucleic acid encoding the antibody or antigen binding fragment thereof, is administered directly to the subject. In some embodiments, antibody or antigen binding fragment thereof is contacted with cells (e.g., immune cells) ex vivo, and the treat cells are administered to the subject (e.g., adoptive transfer). The antibody or antigen binding fragment thereof can enable a more robust immune response to be possible.

### A. Immunotherapy

One aspect of the present invention discloses methods to engineer T cells to make them suitable for immunotherapy purposes.

### 1. Adoptive Immunotherapy

Adoptive immunotherapy, which involves the transfer of autologous antigen-specific T cells generated ex vivo, is a promising strategy to treat viral infections, cancers, inflammation, and autoimmune diseases. The T cells used for adoptive immunotherapy can be generated either by expansion of antigen-specific T cells or redirection of T cells through genetic engineering (Park, Rosenberg et al. 2011). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010).

The current protocol for treatment of patients using adoptive immunotherapy is based on autologous cell transfer. In this approach, T lymphocytes are recovered from subjects, genetically modified or selected ex vivo, cultivated in vitro in order to amplify the number of cells if necessary and finally infused into the subject. In addition to lymphocyte infusion, the host may be manipulated in other ways that support the engraftment of the T cells or their participation in an immune response, for example pre-conditioning (with radiation or chemotherapy) and administration of lymphocyte growth factors (such as IL-2). Each subject receives an individually fabricated treatment, using the subject's own lymphocytes (i.e. an autologous therapy).

Autologous therapies face substantial technical and logistic hurdles to practical application, their generation requires expensive dedicated facilities and expert personnel, they must be generated in a short time following a subject's diagnosis, and in many cases, pretreatment of the subject has resulted in degraded immune function, such that the subject's lymphocytes may be poorly functional and present in very low numbers. Because of these hurdles, each subject's autologous cell preparation is effectively a new product, resulting in substantial variations in efficacy and safety. Ideally, one would like to use a standardized therapy in which allogeneic therapeutic cells could be pre-manufactured, characterized in detail, and available for immediate administration to subjects. Allogeneic cells are obtained from individuals belonging to the same species but are genetically dissimilar. However, studies have found that the use of allogeneic cells presently has many drawbacks. In immune-competent hosts allogenic cells are rapidly rejected, a process termed host versus graft rejection (HvG), and this substantially limits the efficacy of the transferred cells. In immune-incompetent hosts, allogeneic cells are able to engraft, but their endogenous TCR specificities recognize the host tissue as foreign, resulting in graft versus host disease (GvHD), which can lead to serious tissue damage and death. In order to effectively use allogeneic cells, both of these problems must be overcome.

### 2. Adaptive Immunotherapy

Adaptive immune response is a complex biological system where numerous cellular components interact. Professional Antigen Presenting Cells (APC) are able to process foreign bodies and expose them to helper T cells in the context of MHC Class II molecules. Activated helper T cells will in turn stimulate B cells response and cytotoxic T (CTL) cells response. CTL recognize foreign peptides presented by MHC Class I molecules but in the case of alloreactivity, recognize and kill cells bearing foreign MHC Class I. MHC Class I molecules are composed of 2entities: the highly polymorphic, transmembrane heavy chain and a small invariant polypeptide, the β2-microglobuline (β2-m) encoded by B2M gene. The expression of the MHC Class I heavy chain at the cell surface requires its association with the β2-m. Hence, abrogation of β2-m expression in CAR T cells will impair MHC Class I expression and make them invisible to host CTL. However, MHC Class I deficient CAR T cells are susceptibe to lysis by host NK cells, which target cells lacking MHC Class I molecules [Ljunggren H G et al. (1990), Immunl Today. 11:237-244].

NK cells exert cytotoxic functions towards the cells they interact with based on the balance between activating and inhibitory signals they received through different monomorphic or polymorphic receptors. One central activating receptor on human NK cells is NKG2D and its ligands include proteins such as MICA, MICB, ULBP1, ULBP2, ULBP3 [Raulet D H, (2003), Nature Reviews Immunology 3 (10): 781-79]. On the other hand, the inhibitory signal is mediated through the interaction between NK receptors like LIR-1/ILT2 and MHC Class I molecules [Ljunggren H G et al. (1990), Immunl Today. 11:237-244].

### 3. T-cell Mediated Immunity

T-cell mediated immunity includes multiple sequential steps regulated by a balance between co-stimulatory and inhibitory signals that fine-tune the immunity response. The inhibitory signals referred to as immune checkpoints are crucial for the maintenance of self-tolerance and also to limit immune-mediated collateral tissue damage. The expression of immune checkpoints protein can be deregulated by tumors. The ability of tumors to co-opt these inhibitory pathways represents an important mechanism in immune resistance and limits the success of immunotherapy. One of promising approaches to activating therapeutic T-cell immune response is the blockade of these immune checkpoints (Pardoll 2012). Immune checkpoints represent significant barriers to activation of functional cellular immunity in cancer, and antagonistic antibodies specific for inhibitory ligands on T cells including but not limited CTLA4 and programmed death-1 (PD-1) are examples of targeted agents being evaluated in the clinics.

### V. Compositions and Methods of Diagnosing and Staging Feline CDK

Other aspects of the present invention are directed to compositions and methods relating to a rapid and reliable assay for the diagnosis of Feline Chronic Kidney Disease (CDK) used to detect the presence of at least one target indicator in body fluids such as urine. In yet another aspect, the assay according to the present invention is useful to detect CDK in cats with early disease; that is, in cats before the appearance of signs associate with clinical disease. By successfully distinguishing early disease from later CDK accompanied by clinical disease, intervention and treatment may be instituted to limit if not stop progression into clinical CDK and eventual kidney failure.

In one embodiment of an assay according to the present invention, a polymerase chain reaction (PCR) is used in a method to amplify a specific DNA or polynucleotide in vitro, generating thousands to millions of copies of a particular DNA sequence. Detection of FAP DNA or even mRNA is accomplished using body fluids, especially urine, as the sample in the rapid assay. Urine, for example, can be collected from cats using standard methods well known to those in the art, although the optimal collection methods will provide for sterile collection.

As one of ordinary skill will appreciate, reverse transcription PCR (RT-PCR) can also be used in an assay according to the present invention.to diagnose and stage Feline CDK. Using RT-PCR, it is possible to amplify RNA targets by converting the RNA template into complementary (c)DNA using reverse transcriptase, thus providing cDNA to serve as a template for exponential amplification. (see, Bartlett et al., “A Short History of the Polymerase Chain Reaction”, PCR Protocols, 2003). In addition to RT-PCR, there are a number of variations on the basic PCR test including, amongst others, quantitative real-time PCR (qPCR or RT-PCR), allele specific PCR, asymmetric PCR, hot start PCR, multiplex-PCR, nested-PCR, ligation-mediated PCR, intersequence-specific PCR, thermal asymmetric interlaced PCR and touchdown-PCR.

RT-qPCR, or quantitative reverse transcription PCR, combines the effects of reverse transcription and quantitative PCR or real-time PCR to amplify, detect, and quantify a specific target. The process is performed by reverse transcription of total RNA or mRNA to complementary DNA (cDNA) using reverse transcriptase, followed by amplification and detection of specific targets of this cDNA using quantitative PCR (qPCR) or real-time PCR. At each cycle during this PCR, the quantity of DNA can be measures in real-time by using a variety of fluorescent chemistries, including by using either hydrolysis probes such as TaqMan® probes, or a double-stranded DNA binding dye such as SYBR® Green dye. RT-qPCR has a variety of applications including quantifying gene expression levels, validating RNA interference (RNAi), and detecting pathogens such as viruses. The selection of fluorescent chemistry depends upon a variety of factors such as the application, cost, and whether the assay is a singleplex or multiplex assay. DNA-binding dyes are preferred for singleplex, low-throughput assays since they are easier to design, have lesser set-up time, and are more cost-efficient. Fluorescent probes are more commonly employed in high-throughput, multiplex assays that require higher specificity.

Thus, in one embodiment, the present invention provides compositions, methods, and kits for amplifying and/or detecting FAP mRNA isolated from a feline urine sample. The process begins by isolating mRNA from the urine sample and then, using reverse transcription, producing cDNA for subsequent amplification and detection of specific targets using quantitative PCR (qPCR) or real-time PCR. The present invention also contemplates various oligonucleotides useful in the assay with each oligonucleotide recognizing a target sequence within a FAP target region or its complementary sequence. In alternate embodiments of the present invention, one of more of the oligonucleotides may serve as amplification oligomers and/or detection probes for amplification and/or detection of corresponding FAP target nucleic acid. An amplification oligomer is configured to specifically hybridize to a target sequence within a target nucleic acid. At least two amplification oligomers flanking a target region within the target nucleic acid are utilized in an in vitro nucleic acid amplification reaction to generate an amplicon therefrom. Exemplary in vitro amplification reactions include, for example, 10 PCR (e.g., Taqman® PCR) and transcription-associated amplification (e.g., TMA or NASBA). A detection probe, configured to specifically hybridize to a target sequence flanked by at least two amplification oligomers, may be utilized to hybridize specifically to at least a portion of an amplification product, either after completion of or during the amplification process. Methods of the present invention may further may use an oligonucleotide that serves as a capture probe for processing a sample by capturing a FAP target nucleic acid and separating it from other sample components (see, e.g., U.S. Pat. Nos. 6,110,678, 6,280,952, and 6,534,273, each of which is incorporated by reference herein in its entirety).

In another aspect, embodiment of the present invention relates to a method for differentially staging feline CKD, particularly by identifying early CDK even before clinical signs may be present and thus provide the cat parent with the ability to provide supportive care that may ameliorate or slow progression of the disease before significant renal damage occurs. The present invention contemplates identifying early stage disease and differentiating it from later stages by quantifying the amount of FAP target nucleic acid.

In certain embodiments, oligonucleotides and methods of the present invention are useful for amplifying and detecting nucleic acid target sequences present in a sample in a relatively short time so that diagnosis can be made quickly and, in a preferred embodiment, provide an assay able to detect early stage feline kidney disease so that effective treatment can be initiated to limit and perhaps improve the outcome for the cat.

The present invention also contemplates methods for detecting a FAP nucleic acid which, optionally, include a detecting step that uses at least one probe that specifically hybridizes to the FAP amplification product (RNA, DNA amplicon, or cDNA). Accordingly, in certain embodiments, a detection probe of the present invention is configured to specifically hybridize to a region within a target nucleic acid selected from FAP nucleic acid. In certain embodiments, a set of oligonucleotides for detection of FAP mRNA includes two or more detection probes selected from the probes above, whereby the probes are for detecting two or more regions of a FAP target nucleic acid region. In certain embodiments, a detection probe is configured to specifically hybridize to a target nucleic acid region selected from a region within a FAP nucleic acid sequence. In particular variations, a detection probe for detecting an FAP target nucleic acid region is configured to specifically hybridize to a specific region. In some variations, a set of oligonucleotides for detecting FAP target nucleic acid regions includes two or more detection probes selected from the probes above, where the probes are for detecting two or more of an FAP target nucleic acid region.

In particular embodiments, a detection probe as above-configured to specifically hybridize to a target nucleic acid region selected from (a) a region within a FAP nucleic acid sequence. Although these sequences are shown as cDNA sequences, equivalent RNA or RNA/DNA chimeric sequences can be readily derived by the person skilled in the art and are to be considered as falling within the definition of “oligomer” or “detection probe.” In addition, complementary sequences of DNA and RNA and reverse complementary sequences can be readily derived by the skilled person. It is therefore to be understood that a description of any individual sequence of DNA, for example, encompasses its complement, its reverse complement, and equivalent RNA or RNA/DNA chimeric sequences.

Typically, a detection probe in accordance with the pres-ent invention further includes a label. Particularly suitable labels include compounds that emit a detectable light signal, e.g., fluorophores or luminescent (e.g., chemiluminescent) compounds that can be detected in a homogeneous mixture. More than one label, and more than one type of label, may be present on a particular probe, or detection may rely on using a mixture of probes in which each probe is labeled with a compound that produces a detectable signal (see, e.g., U.S. Pat. Nos. 6,180,340 and 6,350,579, each incorporated by reference herein in its entirety). Labels may be attached to a probe by various means including covalent linkages, chelation, and ionic interactions, but preferably the label is covalently attached. For example, in some embodiments, a detection probe has an attached chemiluminescent label such as, e.g., an acridinium ester (AE) compound (see, e.g., U.S. Pat. Nos. 5,185,439; 5,639,604; 5,585,481; and 5,656,744; each incorporated by reference herein), which in typical variations is attached to the probe by a non-nucleotide linker (see, e.g., U.S. Pat. Nos. 5,585,481; 5,656,744; and 5,639, 604, particularly at column 10, line 6 to column 11, line 3, and Example 8; each incorporated by reference herein in its entirety). In other embodiments, a detection probe comprises both a fluorescent label and a quencher, a combination that is particularly useful in fluorescence resonance energy trans-fer (FRET) assays. Specific variations of such detection probes include, e.g., a TaqMan detection probe (Roche Molecular Diagnostics) and a “molecular beacon” (see, e.g., yagi et al., Nature Biotechnol. 16:49-53, 1998; U.S. Pat. Nos. 5,118,801 and 5,312,728; each incorporated by refer-ence herein in its entirety).

A detection probe may further include a non-target-hybridizing sequence. Specific embodiments of such detection probes include, for example, probes that form conformations held by intramolecular hybridization, such as conformations generally referred to as hairpins. Particularly suitable hairpin probes include a “molecular torch” (see, e.g., U.S. Pat. Nos. 6,849,412; 6,835,542; 6,534,274; and 6,361,945, each incorporated by reference herein in its entirety) and a “molecular beacon” (see, e.g., Tyagi et al., supra; U.S. Pat. Nos. 5,118,801 and 5,312,728, supra). Methods for using such hairpin probes are well known in the art.

In particular embodiments, each of one or more detection probes for detecting one or more FAP amplification products includes a fluorescent label (“fluorescent dye compound”). Suitable fluorophores are well-known in the art and include, for example, CalO 560, CalRed 610, and FAM. In some variations of an oligonucleotide set for determining the presence or absence of FAP in the sample, at least one FAP specific detection probe is labeled with a different fluorophore.

In some such embodiments comprising fluorophore-labeled detection probes, the detection probe(s) further include a quencher. Suitable quenchers are well-known in the art and include, for example, BHQ, TAMRA, and DABCLY. A method for determining the presence or absence of FAP, in accordance with the present invention, generally includes the following steps: (1) contacting a sample suspected of containing FAP with at least two amplification oligomers as described above for amplification of the FAP target nucleic acid region; (2) performing an in vitro nucleic acid amplification reaction, where any FAP target nucleic acid, if present in the sample, is used as a template for generating one or more amplification products corresponding to the target nucleic acid present in the sample; and (3) either (i) determining the sequences of the one or more amplification products or (ii) detecting the presence or absence of the one or more amplification products using one or more detection probes as described above for the FAP target nucleic acid regions.

One embodiment of a method according to the present invention generally comprises (a) extracting FAP mRNA from the sample obtained from the subject; (b) using reverse transcriptase reaction to obtain cDNA template; (c) adding the cDNA, a positive control, and optionally a negative control into PCR tubes of PCR reaction system respectively to obtain a corresponding sample reaction tube, positive reaction tube or negative reaction tube, wherein the PCR reaction system contains the primers for detecting FAP (d) performing PCR reaction by placing the reaction tubes on a PCR instrument, setting circulation parameters, and performing PCR reaction; (d) analyzing the results after the PCR reaction is completed; and (e) determining the presence of FAP in the sample; wherein the presence of FAP indicates the presence of fibrosis that precedes renal disease.

In yet another embodiment, a Method according to the present invention will differentiate between stages of CKD, especially in early stages where clinical signs are not evident, by quantifying the amount of FAP present in a sample, wherein the presence of FAP indicates the presence of fibrosis that precedes renal disease.

Yet another aspect of the present invention relates to a PCR diagnostic assay kit for the rapid and reliable diagnosis of CDK in a feline subject comprising (a) primers for detecting FAP from a mRNA sample comprising a forward primer having a nucleotide sequence as set forth in SEQ ID NO: 6 and a reverse primer having a nucleotide sequence as set forth in SEQ ID NO: 7 (b) DNA polymerase; (c) blocking oligonucleotides; (d) a detectable label; and (e) buffers.

In yet another aspect, the invention relates to a PCR diagnostic assay kit for the rapid and reliable diagnosis of CDK in a feline subject comprising (a) primers for detecting Glyceraldehyde 3-phosphate dehydrogenase (GAPDH) from a mRNA sample comprising a forward primer having a nucleotide sequence as set forth in SEQ ID NO: 4 and a reverse primer having a nucleotide sequence as set forth in SEQ ID NO:5 (b) DNA polymerase; (c) blocking oligonucleotides; (d) a detectable label; and (e) buffers. The quantification of GAPDH will be used and a normalization between samples.

Aspects of the present invention are illustrated in the following Examples. Efforts have been made to ensure accuracy with respect to numbers used (e.g., amounts, concentrations, percent changes, and the like) but some experimental errors and deviations should be accounted for. Unless indicated otherwise, temperature is in degrees Centigrade and pressure is at or near atmospheric. It should be understood that these Examples are given by way of illustration only and are not intended to limit the scope of the present invention.

### EXAMPLES

### Example 1: Immunoliposomes for Targeted Therapy

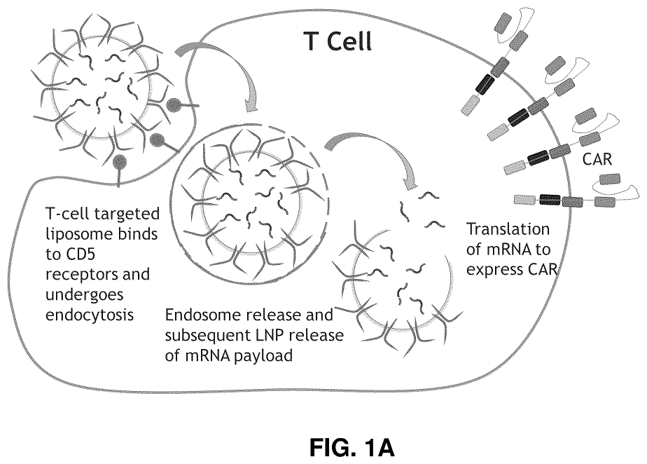

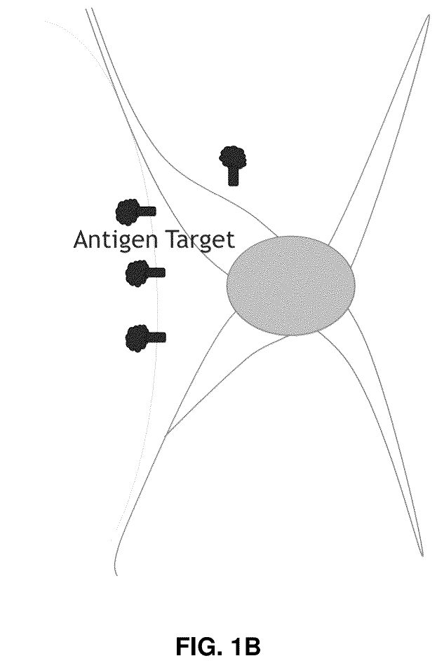

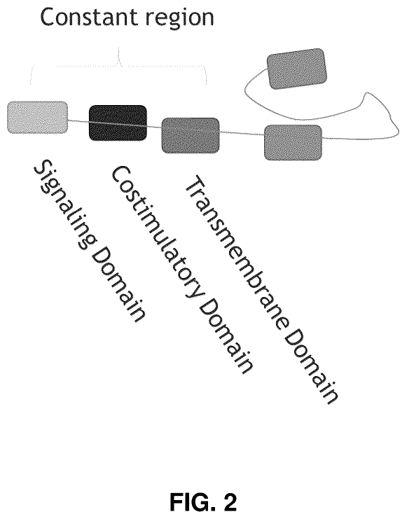

FIGS. 1A-1B illustrate the development of immonoliposomes, wherein the T-cell targeted liposome binds to CD5 receptors and undergoes endocytosis. The endosome is release and subsequently, LNP releases the mRNA payload. This leads to the translation of mRNA to express CAR (FIG. 2). The CAR T cells target cells expressing the target antigen for clearance.

### Example 2: mRNA/LNP to Deliver Transient In Vivo CAR-T Cell

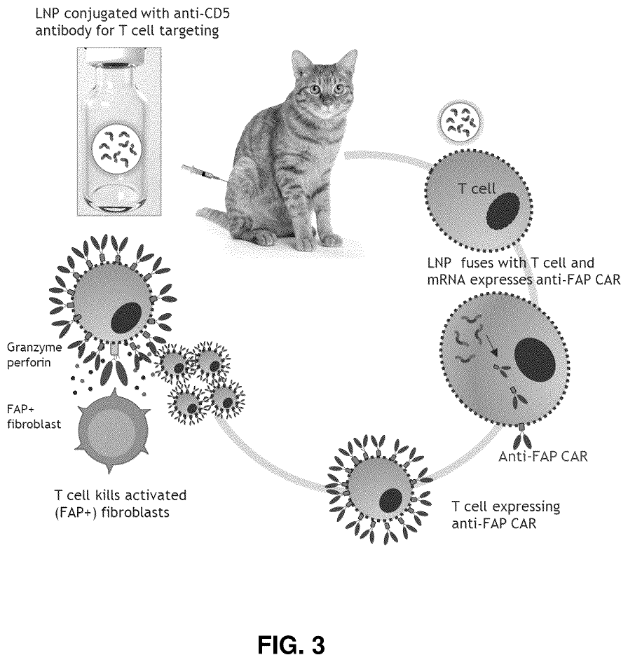

FIG. 3 shows a LNP homing to T cells which then deliver mRNA coding for a chimeric antigen receptor (CAR) that will slow progression of fibrosis. The targeted T cells transiently clear activated fibroblasts, thus limiting progression of renal disease.

### Example 3: Immunoliposomes: A Game Changing Platform for Targeted Therapeutic Development

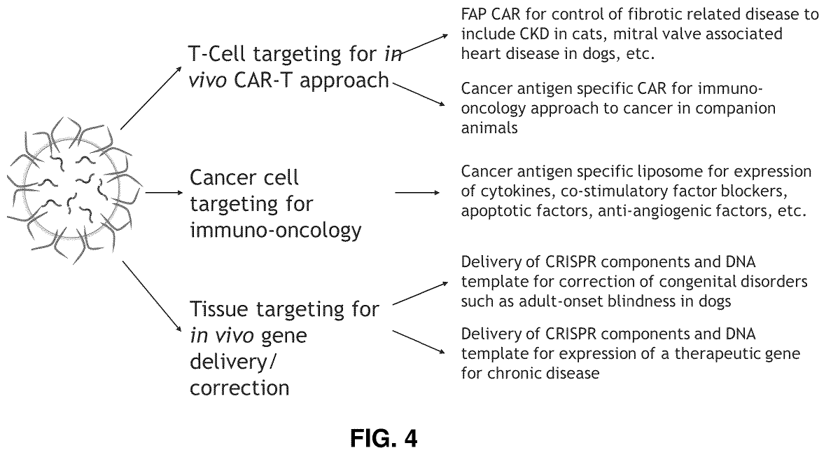

FIG. 4 shows the use of the immunoliposomes as a platform for the development of targeted therapies for different indications. Immunoliposomes can be used in T cell targeting for an in vivo CAR-T approach. They can also be used targeting cancer cells for immunooncology therapeutic approaches. The immunoliposomes can also be used for tissue targeting for in vivo gene delivery/correction.

### Example 4: Feline FAP

Feline FAP will be amplified by RT-PCR from RNA isolated from feline tissues and/or feline fibroblast cell line (AH927 or similar) using primers designed from the Gene bank predicted sequence for the domestic cat (NCBI Reference Sequence: XM_019838169.1, hereby incorporated by reference) which was derived from chromosome C1 (NCBI Reference Sequence: XM_011285347.3, herein incorporated by reference). In vitro expression of feline FAP will be verified by various assays to confirm protein size and function. In addition to the FAP full ORF, the extracellular domain of FAP will be cloned and protein expressed in vitro.

For selection of the FAP gene source, FAP phylogenic trees will be constructed using human, rat, and mouse sequences and compared to the DNA and amino acid sequence of the feline FAP. Several candidate FAP genes from different sources were selected. In addition, we will evaluate consensus sequences (Duperret, E K., et al., Clin Cancer Res 24(5):1190-1201 (2018)). These selected FAP sequences will be assessed for protein expression in vitro. Following determination of the top candidates, a dose response study will be conducted to identify mRNAs with lower doses reaching similar expression levels. The candidate sequences selected are as follows

Sequence of human FAP:
(SEQ ID NO: 1)
ATGGATTGGACCTGGATCCTGTTCCTGGTGGCTGCCGCTACAAGGGTGCACTCCCTC
AGGCCCTCCAGGGTCCACAACTCCGAAGAGAATACTATGAGGGCCCTCACACTGAA
GGATATCCTGAACGGCACATTCTCCTATAAAACCTTCTTTCCAAATTGGATCAGCGG
CCAAGAGTACCTCCACCAGAGCGCCGACAATAACATCGTGCTGTACAATATCGAGA
CCGGACAAAGCTATACAATCCTCTCCAACAGAACCATGAAATCCGTCAATGCCTCCA
ATTATGGACTCTCCCCAGACAGACAATTCGTCTACCTGGAGTCCGACTACTCCAAGC
TCTGGAGATATAGCTATACCGCCACATATTACATTTACGACCTCAGCAACGGCGAAT
TCGTGAGGGGGAACGAGCTCCCCAGGCCCATCCAGTACCTGTGTTGGAGCCCCGTG
GGCAGCAAGCTCGCCTACGTCTACCAGAACAATATCTATCTCAAGCAAAGGCCCGG
CGACCCTCCCTTCCAGATTACCTTTAATGGCAGAGAGAACAAGATCTTTAACGGGAT
CCCTGATTGGGTCTATGAAGAGGAAATGCTGGCCACTAAGTACGCCCTGTGGTGGA
GCCCCAACGGCAAGTTCCTGGCCTACGCTGAGTTTAACGATACAGACATTCCAGTGA
TCGCTTATAGCTACTATGGCGATGAGCAGTATCCCAGAACCATCAACATCCCCTACC
CCAAGGCCGGCGCCAAGAATCCAGTCGTGAGAATTTTCATCATTGACACAACCTACC
CCGCCTACGTCGGCCCCCAGGAAGTCCCTGTGCCTGCTATGATCGCCAGCTCCGACT
ACTATTTCAGCTGGCTCACTTGGGTCACCGATGAGAGAGTGTGCCTCCAGTGGCTCA
AGAGGGTCCAGAACGTCTCCGTGCTCAGCATTTGCGATTTCAGAGAAGACTGGCAG
ACTTGGGATTGTCCCAAAACCCAGGAACACATCGAGGAATCCAGGACCGGCTGGGC
TGGAGGGTTTTTCGTCAGCACCCCAGTCTTCAGCTACGATGCTATCAGCTACTATAA
AATCTTTTCCGATAAAGACGGCTACAAACATATCCACTATATCAAGGATACAGTCGA
GAACGCCATCCAGATCACATCCGGCAAATGGGAGGCCATTAATATTTTCAGAGTGA
CACAGGACAGCCTGTTTTACAGCTCCAACGAGTTCGAAGAGTATCCTGGCAGGAGA
AACATTTATAGAATCAGCATCGGAAGCTATCCACCCTCCAAGAAATGCGTCACATGC
CACCTGAGAAAAGAGAGATGCCAGTATTACACTGCTAGCTTCAGCGATTATGCCAA
GTATTACGCTCTGGTGTGTTATGGGCCTGGAATTCCCATTAGCACTCTGCACGATGG
CAGAACTGACCAGGAAATTAAAATCCTGGAGGAAAACAAGGAACTGGAAAACGCC
CTGAAGAACATCCAGCTGCCCAAAGAAGAGATCAAGAAACTCGAGGTGGACGAGAT
TACTCTGTGGTACAAGATGATTCTCCCACCCCAATTTGACAGAAGCAAGAAATATCC
CCTCCTGATCCAGGTCTACGGCGGGCCCTGTTCCCAGAGCGTGAGGAGCGTGTTCGC
CGTGAACTGGATTTCCTACCTGGCCTCCAAGGAAGGAATGGTGATCGCCCTGGTGGA
CGGAAGAGGAACCGCCTTCCAAGGGGATAAACTCCTGTACGCCGTGTATAGAAAAC
TGGGGGTGTACGAAGTCGAGGATCAGATCACTGCCGTGAGGAAGTTTATCGAAATG
GGCTTCATTGACGAGAAGAGGATCGCCATTTGGGGCTGGGCTTACGGAGGCTACGT
GTCCAGCCTCGCTCTGGCTAGCGGCACCGGGCTCTTTAAGTGCGGGATCGCCGTGGC
CCCCGTCTCCAGCTGGGAGTATTACGCTAGCGTGTACACAGAAAGATTCATGGGCCT
GCCAACAAAAGATGACAATCTGGAACACTATAAGAATAGCACTGTCATGGCTAGGG
CTGAGTACTTTAGGAACGTCGATTATCTCCTGATTCACGGCACTGCTGACGATAACG
TGCACTTCCAGAACTCCGCCCAGATTGCTAAAGCTCTCGTGAACGCTCAGGTGGACT
TCCAGGCTATGTGGTACTCCGACCAAAATCATGGGCTCAGCGGACTCAGCACAAAC
CACCTGTATACCCACATGACTCATTTTCTGAAACAATGTTTTTCCCTGAGCGACTAAA.
Sequence of chicken FAP
(SEQ ID NO: 2)
ATGGATTGGACATGGATTCTGTTCCTGGTGGCCGCTGCCACCAGAGTGCACAGCCTC
CTGCCCTCCAAAGTCGTGACAACTGTGGACGGCCCAAGGGCTCTCACCCTCGATGAC
TATCTCAACGGAAACTTTCAGTACAAGACATATTTCCCCTATTGGGTCTCCGATTCCG
AATACCTCCACCAGAACCAGGAAGATGACATTATCCTCTTCAATGTGGACATGAATT
ACCTCACTACCATCATGACCAACTCCACCATGAAGCAGGTGAACGCCAGCAATTAC
GTGATGAGCTCCGACAAGTATTTTATCGCTCTGGAAAGCAATTATTCCAAGCTCTGG
AGATATAGCTACACCGCCAGCTATCACATTTATGATCTCATCTACGGAGGCTTCGTG
ACCGAAAATCAGCTGCCCCACAAAATTCAGTACATTTCCTGGAGCCCCGTCGGCCAC
AAGCTGGCTTACGTCTACCAGAACAATATCTACCTCAAACAAAGCCCCAGAGAGGC
CCCAATTAAACTCACCTCCGACGGCAAAGAAAATGAAATCTTTAACGGAATCCCTG
ATTGGGTGTACGAAGAGGAAATGCTGGCTACCAAATACGCCCTCTGGTGGAGCCCA
AGCGGGAAATACCTCGCCTACGTGCAGTTTAACGACTCCGATATTCCAGTGATTGAG
TATTCCTACTTCGGAGAGGACCAGTACCCCAGAAAAATTATCATTCCATATCCTAAG
GCCGGAGCTAAGAACCCTACCGTGAAAGTGTTCATCGTCGACACTACAAACATCGA
AGCCTTTGGCCCTAAAGAAGTCCCTGTGCCAGCCGTGATCGCTTCCAGCGACCACTA
TTTCACCTGGCTGACCTGGGTGACCGACTCCAGAGTCGGCGTGCAATGGCTGAAGAG
GATTCAGAACTTCTCCGTCCTGGCCATCTGCGACTTCAAAGAAAACAGCAACACATG
GGACTGTCCCGAAAACCAGCAACATATTGAAGAGTCCCAAACAGGCTGGGCTGGAG
GGTTTTTCGTCTCCGCCCCATATTTTACATCCGATGGCAGCTCCTATTACAAGATCTT
CAGCGACAAGAACGGGTATAAACACATCCACTACATCAATGGCAGCGTCGAAAACG
CCATCCAGATTACCAGCGGCGAATGGGAAGCCATTTACATTTTCAGAGTGACCAACG
ATGCCATTTTCTATAGCTCCAACGAATTTGAGGGGTATCCCGGCAGGAGAAACATCT
ACAAAATTAGCATCGGATCCAAGCCAATTAGAAAGCTGTGCATCACTTGTAATCTCA
GGAAGGAAAGGTGTCAGTATTACACCGCCAGATTCAGCGAGAGGAGCAAGTATTAC
GCCCTGATCTGCTACGGGCCTGGCATTCCTATCAGCACCCTGTTTGAGACAGAAAGC
GACAGAGAACTCAGAATCCTGGAAGACAACCAGGAGCTCCAGTCCGCCCTGCAAGA
GATCATTCTGCCCAAGGAAGAGATTAACAAACTCGAGGTGGACGGAATCACCCTGT
GGTATAAGATGCTGATTCCCCCTCAATTCGATAGGAGCAAAAAGTACCCACTGCTCA
TCCAGGTGTATGGGGGCCCATGCTCCCAGAATGTCAAGCACACATTCAGCATCAGCT
GGATTACATACCTGGCCAGCAAAGAAGGGATTATCGTCGCCCTGGTGGACGGAAGA
GGCACCGCCTACCAGGGAGATAAGATCCTGCACGCCGTCTATAGAAGGCTCGGGGT
GTACGAAGTCGAGGACCAAATTTCCGCCGTGAAGAAATTCATTGAGATGGGCTTCAT
TGATGAAAAGAGAATCGCTATCTGGGGCTGGGCTTATGGGGGCTATGTCACCTCCCT
GGCCCTGGGCAGCGGGTCCGGCGTGTTCAAATGCGGCATCGCTGTGGCCCCCGTGTC
CAGCTGGGAGTACTATGCCAGCATCTATACTGAAAGGTTTATGGGCCTCCCCGTGGA
GAGCGATAACCTGGAACACTATAAAAACAGCACAGTGATGGCTAGAGCCAAAAACT
TCCAAAATGTGGAATACCTCCTGATCCACGGGACTGCCGATGACAACGTCCACTTCC
AAAATAGCGCCCAGATCGCCAAGGCCCTGGTCAACGCTCAGGTGGACTTTCAAGCC
ATGTGGTACACAGATCAGAACCACGGCATTCCCGGGCTCAGCTCCAAGCATCTCTAT
ACCCACATGACTCACTTCCTGAAGCAGTGCTTCAGCCTGAGCGAATAAA.
Sequence of Consensus FAP
(SEQ ID NO: 3)
ATGGATTGGACCTGGATTCTCTTTCTGGTCGCCGCTGCCACTAGAGTGCACAGCCTC
AGGCCTAGCAGGGTGCACAACAGCGAAGGGAACACCACTAGGGCCCTCACCCTGAA
GGACATCCTGAATGGGACATTCAGCTACAAGACCTTCTTTCCTAATTGGATCAGCGG
CCAGGAGTATCTCCACCAATCCACCGACAATAACATCGTGCTCTACAATATTGAGAC
CGGGGAATCCTATACCATTCTCAGCAACTCCACAATGAAATCCGTGAACGCCAGCA
ATTACGGACTGTCCCCCGACAGGCAATTCGCTTATCTGGAGTCCGACTACTCCAAGC
TGTGGAGGTATAGCTATACTGCCACTTACCACATTTATGATCTGTCCAATGGCGAAT
TTGTGAGAGGGAACGAGCTGCCCAGACCAATCCAGTACCTGTGCTGGAGCCCAGTG
GGGAGCAAGCTCGCTTACGTCTACCAGAACAATATTTATCTGAAACAGAGACCCGA
AGACCCACCCTTTCAGATCACCTATAATGGCAGGGAGAACAAGATTTTTAACGGCAT
CCCCGACTGGGTGTACGAAGAGGAAATGCTGGCCACCAAATACGCCCTCTGGTGGT
CCCCTAACGGCAAATTCCTGGCTTATGCCGAGTTCAACGATACCGACATCCCTGTCA
TCGCCTATAGCTATTACGGAGACGAGCAGTACCCTAGAACCATCAATATTCCCTACC
CAAAAGCCGGCGCCAAAAACCCCGTCGTGAGGATCTTTATTATCGACACCACATAC
CCAGAGCACGTGGGACCCAGGGAGGTCCCCGTGCCTGCTATGATTGCCTCCAGCGA
CTACTATTTTTCCTGGCTCACCTGGGTGACCGACGAAAGGGTGTGCCTGCAGTGGCT
GAAAAGGATCCAGAACGTGTCCGTGCTCAGCATCTGCGATTTCAGGGAGGATTGGC
AGACCTGGGATTGTCCTAAGACACAGGAACACATTGAAGAGAGCAGAACAGGCTGG
GCCGGCGGATTCTTTGTGTCCACCCCCGTGTTTTCCTATGACGCTATTAGCTATTACA
AAATCTTCTCCGATAAGGATGGCTATAAACATATTCACTACATCAAGGACACTGTGG
AGAATGCCATTCAGATTACTAGCGGCAAGTGGGAGGCCATTAACATCTTCAGAGTC
ACCCAGGATAGCCTGTTCTACTCCAGCAACGAGTTTGAGGGATACCCCGGCAGGAG
AAACATCTACAGGATTAGCATCGGCAGCTATCCCCCATCCAAGAAATGCATCACAT
GTCATCTGAGGAAGGAGAGATGCCAGTACTATACAGCCAGCTTCTCCGACTATGCTA
AATATTACGCCCTGGTGTGTTATGGCCCTGGCCTCCCAATCTCCACTCTGCATGACG
GGAGAACTGACCAAGAGATCAAGATTCTGGAGGAAAACAAGGAGCTGGAAAACGC
TCTGAAGAATATCCAACTGCCCAAGGAAGAGATCAAGAAACTGGAAGTGGATGAAA
TTACCCTGTGGTACAAAATGATCCTGCCTCCCCAATTCGATAGATCCAAAAAGTACC
CCCTCCTGATCCAAGTGTACGGGGGCCCTTGCAGCCAGTCCGTGAGGTCCGTGTTTG
CCGTCAACTGGATCTCCTACCTGGCCAGCAAGGAAGGGATCGTGATTGCCCTGGTGG
ATGGCAGAGGCACAGCTTTCCAGGGAGATAAACTCCTGTACGCCGTCTATAGAAAA
CTCGGCGTGTACGAGGTGGAGGACCAGATTACCGCCGTGAGGAAGTTTATCGAGAT
GGGCTTCATCGATGAGAAAAGGATCGCCATTTGGGGGTGGGCTTACGGAGGGTACG
TGTCCAGCCTCGCCCTGGCCAGCGGAACAGGGCTGTTCAAGTGCGGGATCGCCGTC
GCCCCTGTGAGCTCCTGGGAATATTACGCCTCCATCTACACCGAAAGGTTCATGGGC
CTGCCCACTAAGGATGACAATCTGGAGCACTACAAGAACTCCACTGTCATGGCCAG
GGCTGAGTACTTCAGAAATGTGGACTACCTCCTGATCCATGGCACAGCTGACGATAA
CGTGCATTTTCAGAACAGCGCCCAGATTGCCAAAGCCCTGGTGAATGCCCAGGTGG
ATTTCCAGGCCATGTGGTATAGCGACCAGAACCACGGAATCAGCGGCCTCAGCACC
AAACACCTGTATACCCATATGACCCACTTCCTGAAGCAGTGTTTTTCCCTGAGCGAT
TAAA.

### Example 5: Generation of Feline Anti-FAP Hybridoma for Identification of Vh and Vl

To create a FAP CAR, first a specific feline anti-FAP antibody must be generated so the scFv fragment can be reverse engineered. FAP-null mice will be immunized intraperitoneally and boosted twice with FAP and/or the extracellular domain of FAP. After the final boost, splenocytes will be harvested and fused to myeloma cells. Hybridomas will be screened for specific binding to FAP, and/or the extracellular domain of FAP and/or activated primary wildtype fibroblasts. Lack of binding will be accessed to the empty expression vector and/or activated primary fibroblasts from FAP-null mice. Selected monoclonal antibodies will be further characterized by various assays for specific binding to feline FAP and the extracellular domain of FAP as well as determination of affinity and antibody isotype.

Using standard protocols for the production and screening of hybridomas well known by those of skill in the art (see, for example, Molecular Cloning: A Laboratory Manual (Fourth Edition), Volume 1, 2 & 3 4th Edition, Michael R. Green and Joseph Sambrook, 2012), laboratory strain mice were immunized intraperitoneally and boosted twice with a recombinant FAP and/or the extracellular domain of FAP. After the final boost, splenocytes were harvested and fused to myeloma cells. Hybridomas were screened for specific binding to FAP, and/or the extracellular domain of FAP, by ELISA and/or activated primary wildtype fibroblasts by immunocytochemistry. Lack of binding was accessed to the empty expression vector and/or activated primary fibroblasts from FAP-null mice. Selected monoclonal antibodies will be further characterized for on/off kinetics and binding affinities using Octet biosensors. Epitope binning will be performed to ensure selected antibodies have different epitopes.

### Example 6: Development of FAP CAR and Confirmation of T Cell Activation

The feline FAP mouse hybridoma will be reverse engineered to determine the sequence of the Vh and Vl regions. Total mRNA will be extracted from the hybridoma and RT-PCR will be used to clone these regions into a plasmid. Various clones will be sequenced and correct alignment verified. Selected clones will then be cloned into a lentivirus vector containing the T cell signaling proteins. Primary feline T cells will be transduced with the lentivirus FAP CAR and the FAP CAR-T cell will be accessed for activation of T cells using real time cell analysis and cytokine assays.

### Example 7: Generation of mRNA Expressing Anti-FAP CAR and Encapsulation in LNP

The FAP CAR sequence will then be cloned into an expression vector using a T7 promoter for mRNA production. Briefly, MEGAScript T7 kit (Invitrogen AMB13345) will be used to produce mRNA using m1Ψ-5′-triphosphate (TriLink N-1081) and the polyA tail will be 101 nucleotide-long. CleanCap (TriLink, San Diego, CA) will be used to cap in vitro transcribed mRNAs. Purified mRNA will be encapsulated in LNPs using the Ignite instrument from Precision NanoSystems (Vancouver, CA) which will enable the controlled and precise assembly of LNPs using microfluidic technology. The particle size, size distribution and encapsulation efficiency of RNA loaded particles will be characterized using a particle size analyzer and a Ribogreen assay.

### Example 8: Development of Anti-CD5 Antibodies for LNP Homing to T Cells

Feline specific CD5 (or similar) hybridomas or aptamers will be generated, screened and validated for the ability to bind and activate primary feline T cells. Assays will be run to verify that the generated anti-CD5 antibody activates T cells.

### Example 9: CD5 Homing LNPs Loaded with mRNA Expressing Anti-FAP CAR

The anti-CD5 antibody will be conjugated to LNPs using SATA (N-succinimidyl S-acetylthioacetate)-maleimide conjugation chemistry (Tombacz et al). The LNPs will be modified with DSPE-PEG-maleimide micelle while the antibody will be modified with SATA to introduce sulfhydryl groups allowing conjugation to maleimide. The SATA on the antibody will be deprotected using 0.5 M hydroxylamine and purified using chromatography. Using thioether conjugation chemistry, the antibody's reactive sulfhydryl group will be conjugated to the LNPs' maleimide component and purified by chromatography. Stability of the conjugated antibody to the LNP will be evaluated.

### Example 10: In Vitro Validation of Cell Killing Activity of Anti-FAP CAR T Cells

To verify in vitro LNP homing to CD5+T cells, CD5 specific antibody conjugated to LNPs containing mRNA encoding either FAP CAR or green fluorescent protein (GFP) will be incubated with feline T cells and the percent of T cells expressing either GFP or FAP CAR will be measured by flow cytometry. Similarly, control LNPs conjugated with antibody that is not specific for T cells will be loaded with either GFP or FAP CAR and measured for mRNA expression.

To verify the interaction of the FAP CAR T cells with FAP, HEK293T cells or similar overexpressing red fluorescence protein (RFP)—tagged FAP will be used to observe the transfer of plasma membrane fragments from the EAP cell to the T cell or trogocytosis. Comparative studies will be run using either a lentivirus-engineered FAP CAR T cells or CD5/LNP/FAP CAR T cells. Trogocytosis the lack there of will be observed with live-imaging confocal microscopy.

To verify that the anti-FAP CAR T cells kill FAP expressing cells, T cells described above will be co-cultured with HEK293T cells expressing feline FAP and luciferase. After overnight incubation, cells will be washed, lysed and assayed for luciferase, Reduction of luciferase activity will indicate killing of FAP expressing cells.

### Example 11: In Vivo Validation and Dose Response and Safety of CD5/LNP Homing to T Cells

Cats will be injected intravenously with varying doses of CD5/LNP expressing luciferase mRNA and splenic T cells will be evaluated for luciferase activity compared to similar LNPs conjugated with a control antibody. Other organs will be evaluated for luciferase expressing T cells, specifically the liver and kidney cells as well as urine. Safety will be assessed by clinical signs as well as gross lesions and histopathology.

### Example 12: Validate FAP Upregulation in CKD Cats

Histopathological tissues from CKD cats at the four different stages of the disease will be evaluated for the upregulation of FAP using assays to measure concentrations of both FAP RNA and FAP protein. These data from upregulated FAP in diseased tissues will be compared to matching normal tissues and the correlation coefficient will be determined.

### Example 13: Determine the Effective Dose of a mRNA/LNP CAR

Cats with at least one parameter listed in Table 1 will be recruited to determine the effective dose of CD5/LNP/FAP CAR. Test and treatment control groups will be measured for at least one parameter in Table 1 and followed for approximately one year. Effective doses will be determined by a statistical and/or clinical difference between the test group having less progression of disease defined by the international Renal Interest Society compared to the treatment control group.

TABLE 1
Parameters

Upregulation of fibroblast activation protein (FAP)
Elevated Symmetric dimethylarginine (SDMA) concentration
Increase in creatinine levels
Increased Blood urea nitrogen (BUN)
Increased BUN/creatinine ratio
Azotemia
Elevated phosphorous
Proteinuria
Decreased urine specific gravity (USG) or isosthenuria
Decrease in glomerular filtration rate (eGFR)
Abnormalities in an ultrasound imaging of kidney
Renal biopsy histopathologic lesions
Clinical signs such as depressed appetite, lethargy, gingivitis,
oral ulcers, dehydration, weight
loss, poor hair quality, anemia and/or Polyuria/Polydipsia (PU/PD)

### Example 14: Development of an Allogeneic CAR-T Ex Vivo

Universal, allogeneic CAR-T cells will be engineered to no longer express endogenous T cell receptor (TCR) andior major histocompatibility complex (MHC) molecules, thereby preventing graft-versus-host disease (CBVHD) or rejection, respectively. Self-driving CARs co-express a CAR and a chemokine receptor, which binds to a tumor ligand, thereby enhancing tumor homing. CAR-T cells engineered to be resistant to immunosuppression (Armored CARs) will be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling. A self-destruct CAR may be designed using RNA delivered by electroporation to encode the CAR. One or more of these methods will be used to generate an allogeneic anti-FAP CAR-T cell for determination of safety and efficacy studies in cats.

### Example 15: Methods for Development of Anti-Feline FAP Monoclonal Antibodies

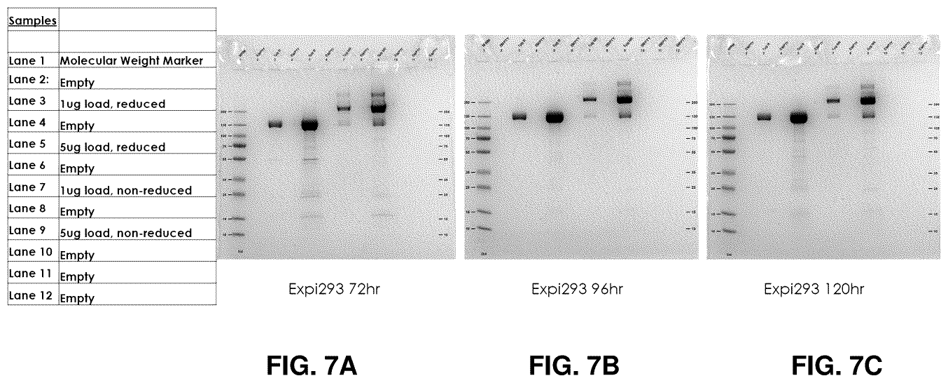

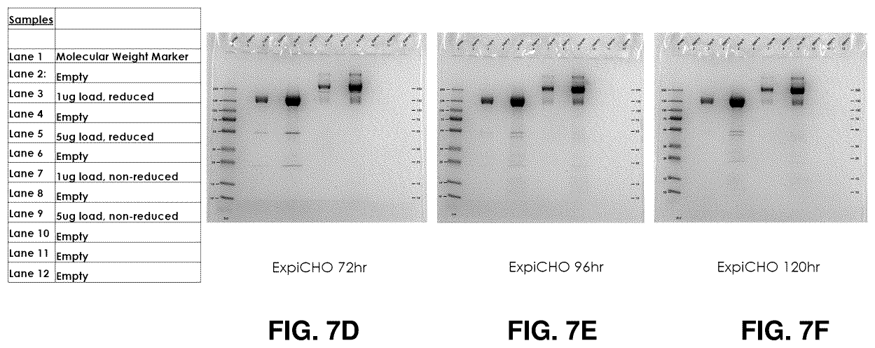

Gene Synthesis and Cloning & Virus Generation: Baculovirus expression of fFAP-HIS: The designed sequence(s) were synthesized genes and cloned into the pv11392 insect expression vector using standard methods. Co-transfection of the purified, recombinant plasmid and linearized baculovirus DNA (BD BaculoGold™; BestBac etc.) was performed to generate bacmid DNA to transfect Sf9 cells to prepare p0 virus stock. The p0 supernatants (5 mL) were harvested used to produce passage 1 (p1) to generate 50 ml of high-titer baculovirus stock (HTS). The pl virus stock was tittered and tested for mycoplasma. A time-course of protein expression was performed to expression of fFAP in serum-free cultures of Sf9 and T. ni cells at the 3×50 mL scale using an appropriate MOI for each cell line. Supernatant was harvested (1×50 ml flask Sf9 and T. ni) to assess expression at timed intervals to evaluate the integrity, stability and optimum yield of the protein from gene expression by SDS & Western Blot. The supernatant from each individual flask was purified separately using Protein IMAC affinity resin. The final product was dialyzed into a biological buffer (such PBS pH 7.2-7.4). Purity was demonstrated by loading 1 microgram and 5 micrograms (both reduced and non-reduced) on a 4-20% Tris-glycine SDS PAGE gel (FIGS. 7A-7F). The gel was stained with Coomassie based stain and the gel image was scanned to provide a percent purity value.

Gene Synthesis and Mammalian Cell Expression/Purification: An N-terminal Fc fusion expression plasmid with appropriate signal sequences that expresses fFAP was generated. PEI based transfection reagents was used to transfect and expand 3×50 ml expressions in HEK293 an CHO-s cell lines for serum-free suspension culture. Supernatant was harvested (1×50 ml flask CHO & HEK293 expression) at timed intervals (72, 96 & 120 hours) to evaluate the integrity, stability and optimum yield of the protein from gene expression by SDS & WB. The supernatant from each individual flask was purified separately using Protein A affinity resin. The final product was dialyzed into a biological buffer (such PBS pH 7.2-7.4). Purity was demonstrated by loading 1 microgram and 5 micrograms (both reduced and non-reduced) on a 4-20% Tris-glycine SDS PAGE gel (FIGS. 2a and 2b for HEK293 and CHO respectively). The gel was stained with Coomassie based stain and the gel image was scanned to provide a percent purity value. Using this method, mg quantities of hFc-fFAP were produced with >94% purity (Table 2). The 120 hr HEK293 lot of hFc-fFAP was utilized for immunizations.

TABLE 2

| | | Conc. | Volume | Yield | Purity by SDS |
|---|---|---|---|---|---|
| Sample | Lot# | (mg/ml) | (mL) | (mg) | PAGE |
| Expi293, 72 hr | KP17409 | 0.49 | 2.63 | 1.28 | >95% |
| Expi293, 96 hr | KP17410 | 1.53 | 3.55 | 5.44 | >95% |
| Expi293, 120 hr | KP17411 | 1.63 | 3.89 | 6.35 | >95% |
| ExpiCHO, 72 hr | KP17412 | 0.43 | 2.17 | 0.94 | >94% |
| ExpiCHO, 96 hr | KP17413 | 0.69 | 2.67 | 1.83 | >97% |
| ExpiCHO, 120 hr | KP17414 | 0.61 | 3.17 | 1.92 | >97% |

### Example 16: Mouse Immunization and Hybridoma Development

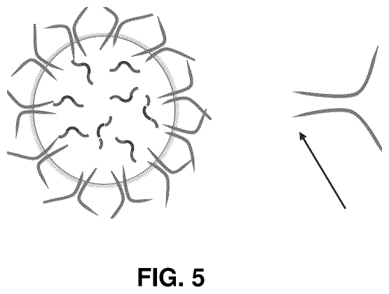

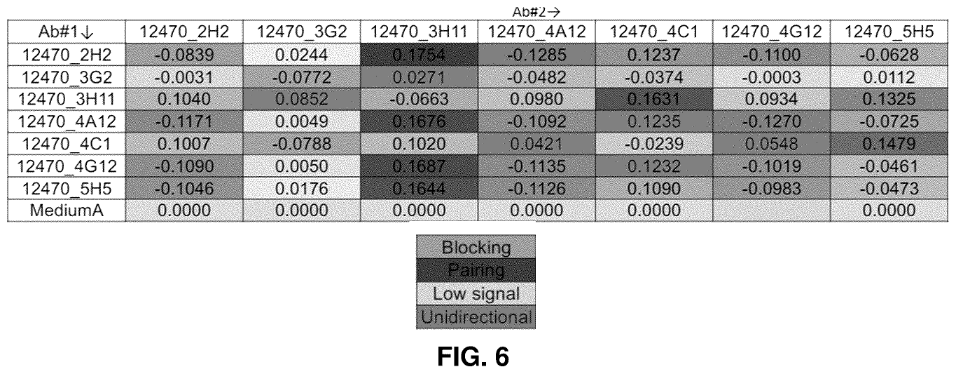

mAb development to Feline Prolyl Endopeptidase FAP using hFc-fFAP fusion protein as an immunogen was performed. Specifically, 3 SJL strain of mice were immunized to include subsequent boosts with hFc-fFAP protein. Tail bleeds were performed for serum isolation at established timepoints post prime and boost regimen. To assess serum titers of anti-fFAP antibodies, 100 ng of hFc-fFAP antigen was coated per well of a 96-well plate in sodium bicarbonate buffer, pH 9.6. Serum dilutions at 1:1000, 1:4000, 1:16000, 1:64000 and 1:256000 were tested as primary Ab. Naïve serum at 1:1000 dilution was used as a background control. The secondary Ab for the serum was an HRP-Goat anti-mouse IgG gamma chain specific Ab followed by detection with TMB substrate and absorbance reading at 620 nm OD. Strong serum titers greater were observed in the serum of the immunized mice (data not shown). Because hFc-fFAP was used as the immunogen, serum samples were tested again against the His-tagged fFAP (Insect Cell Hi Five Expressed protein from the Test Expression 96 hour purified protein) to check the specific titer to Feline FAP. This analysis confirmed titers of anti-fFAP anitbodies were present in the immunized mice. Based on the titer analysis, one of the three mice was selected for electrofusion for hybridoma development and clone selection. Antigen specific clones were identified using ClonePix FL based onshape, size and fluorescence intensity. High throughput ELISA screening of hybridoma candidates was performed as described above by ELISA with both hFc-fFAP, fFAP-HIS and pooled human IgG protein. Of the 20 clones analyzed, 9 were identified to express a monoclonal antibody specific to fFAP (Table 3). On/off rate and KD ranking at a single analyte concentration was performed using standard Octet biosensors to generate Octet Run Time and Affinity Data (Table 4). Octet based epitope binning using an unlabeled sandwich immunoassay was used to determine differential epitope binding between the clones. In the complete 7×7 epitope binning assay, 3 different bins were identified (FIG. 6): Bin1:5H5, 4G12, 2H2, 4Al2. These share a common epitope region and block each other. Bin2: 4C1, pairs with Bin1 and 3H11. And when 4G12, and 4A12 are used as capture antibodies, 4C1 works well as detection antibody or Ab2. Thus, 4C1 shows unidirectionality with 4G12 and 4A12. Bin3: 3H11, pairs with Binl and 4C1. In particular, strong signals were observed when clones from Binl were used as capture and 3H11 for detection Bin4: 3G2 epitope bin is ambiguous due to poor antigen capture leading to low Ab2 (detection) signal in general.

TABLE 3

| Clone# | Ag1 | Ag2 | Ag3 |
|---|---|---|---|
| 1A1 | 3.267 | 0.075 | 0.046 |
| 1B7 | 2.387 | 0.061 | 0.066 |
| 1D2 | 2.588 | 0.070 | 0.065 |
| 2B7 | 2.125 | 0.074 | 0.048 |
| 2H2 | 2.431 | 1.571 | 0.054 |
| 3B10 | 2.088 | 0.059 | 0.085 |
| 3G2 | 2.979 | 2.144 | 0.045 |
| 3H11 | 2.720 | 0.651 | 0.059 |
| 4A12 | 2.799 | 2.748 | 0.043 |
| 4B9 | 2.543 | 0.312 | 0.106 |
| 4C1 | 2.905 | 1.365 | 0.046 |
| 4D8 | 2.358 | 0.060 | 0.162 |
| 4F8 | 3.366 | 0.191 | 0.044 |
| 4G12 | 3.377 | 3.170 | 0.045 |
| 4G9 | 2.157 | 0.112 | 0.210 |
| 5D6 | 2.157 | 0.243 | 0.043 |
| 5F5 | 2.144 | 0.075 | 0.045 |
| 5G10 | 2.046 | 0.128 | 0.061 |
| 5G6 | 1.949 | 0.049 | 0.099 |
| 5H5 | 3.197 | 2.270 | 0.044 |

TABLE 4

| Loading | | | | | |
|---|---|---|---|---|---|
| Sample | | KD | ka | kdis | Full |
| ID | Response | (M) | (1/Ms) | (1/s) | R\hat{ }2 |
| 12470_2H2 | 0.4488 | 4.76E−10 | 3.31E+05 | 1.58E−04 | 0.9942 |
| 12470_3G2 | 0.2546 | 1.65E−09 | 1.33E+05 | 2.20E−04 | 0.998 |
| 12470_3H11 | 0.3823 | 1.69E−09 | 1.12E+05 | 1.89E−04 | 0.9989 |
| 12470_4A12 | 0.4584 | 3.71E−10 | 2.98E+05 | 1.10E−04 | 0.9935 |
| 12470_4C1 | 0.3107 | 1.10E−09 | 1.78E+05 | 1.95E−04 | 0.9954 |
| 12470_4F8 | 0.1681 | 5.79E−09 | 4.67E+04 | 2.70E−04 | 0.9906 |
| 12470_4G12 | 0.4101 | 2.53E−10 | 3.09E+05 | 7.80E−05 | 0.9944 |
| 12470_5D6 | 0.3501 | 1.56E−09 | 4.07E+05 | 6.32E−04 | 0.989 |
| 12470_5H5 | 0.368 | 2.45E−10 | 3.07E+05 | 7.52E−05 | 0.9951 |

### Example 17: Collection of Urine from Cats

Urine will be isolated from cats using any method suitable for collecting sterile urine sample such as collection via cystocentesis in the morning prior to void, when the bladder is full to remove urine directly from the bladder. Briefly, cystocentesis involves insertion of a needle, with a 6- or 12-mL syringe attached, through the cat's abdominal wall and bladder wall to obtain the urine sample (Diagnostic and Therapeutic Procedures, 2012, Richard B. Ford and Elisa Mazzaferro, in Kirk & Bistner's Handbook of Veterinary Procedures and Emergency Treatment (Ninth Edition)). Alternatively, urine can be collected via catheter.

### Example 18: Isolation of mRNA from Urine and RT-PCR

Detection of particular mRNA species from urine has been used previously for biomarker detection for renal fibrosis as well as other renal pathophysiological conditions (Liquid biopsy biomarkers of renal interstitial fibrosis based on urinary exo some—PubMed (nih.gov)). Urine samples will be collected as above and RNA isolated using a Qiagen RNeasy FFPE kit (cat#73504) for RNA isolation. RNA will be quantified with the nanodrop. RNA will be added to SS3 first strand synthesis kit and used to produce cDNA following the instructions of the kit in 8-tube strips. 100 uM primer solutions will be made following IDT instructions for dilution. Master mixes will be prepared for both gapdh and EAP by multiplying volume by number of samples and combining SYBR master mix, H2O, forward primer, rand everse primer (primers listed in Table 1 below). After determining plate set up, using triplicates of both gapdh and FAP, the Master mix for each primer set will be added to each well with the corresponding gene for amplification. cDNA samples from each isolate will be added in triplicate for both gapdh and FAP primer sets. PCR plates will be covered with a sealing film, centrifuged at 2,000G for 1 minute to mix and settle samples and then placed in the PCR machine, PCR will be performed using the Quant Studio 3/Applied Biosy stems to perform a comparative run with melt. PCR data will be preprocessed in Thermo cloud to develop an amplification plot for each sample (both for gapdh and FAP). The threshold value of both gapdh and FAP will be adjusted to match and be approximately halfway up the linear segment of amplification. After exporting the processed data in a csv file, the Cq values will be analysed for each sample in triplicate. The dCT will be calculated by subtracting the HK Cq avg from the GOI average. The ddCt baseline will be calculated using control samples by averaging their dCt. The ddCt will be calculated by subtracting the control baseline from dCt for each sample. From that the 2\hat{ }-ddCt will be calculated to obtain relative gene expression for FAP in the sample. The sequences of gapdh forward and reverse primers and feline FAP forward and reverse primers are as follows:

TABLE 5

| | | |
|---|---|---|
| Feline gapdh forward | 5′-AAG GTG TGA AGG TCG GAG T-3′ | SEQ ID |
| primer | | NO: 4 |
| Feline gapdh reverse | 5′-GAT GAC AAG TTT CCC GTT CTC A-3′ | SEQ ID |
| primer | | NO: 5 |
| Feline FAP forward | 5′--GAA GTT GAG GAC CAG ATC ACA GC-3′ | SEQ ID |
| primer | | NO: 6 |
| Feline FAP reverse | 5′-CAT AGC CTC CGT AGG ACC AGC-3′ | SEQ ID |
| primer | | NO: 7 |

### Example 19: Study Validating FAP in Feline Fibrosarcoma and Presence of Kidney Disease

From a bank of tissues, tissues from feline cutaneous fibrosarcomas were used to evaluate the up-regulation of FAP. Immunohistochemistry was performed on feline tissues using the cross-reactive monoclonal antibody (ab207178 from abcam). Utilizing this antibody, formalin fixed, paraffin embedded feline tissue sections were analyzed by immunohistochemistry.

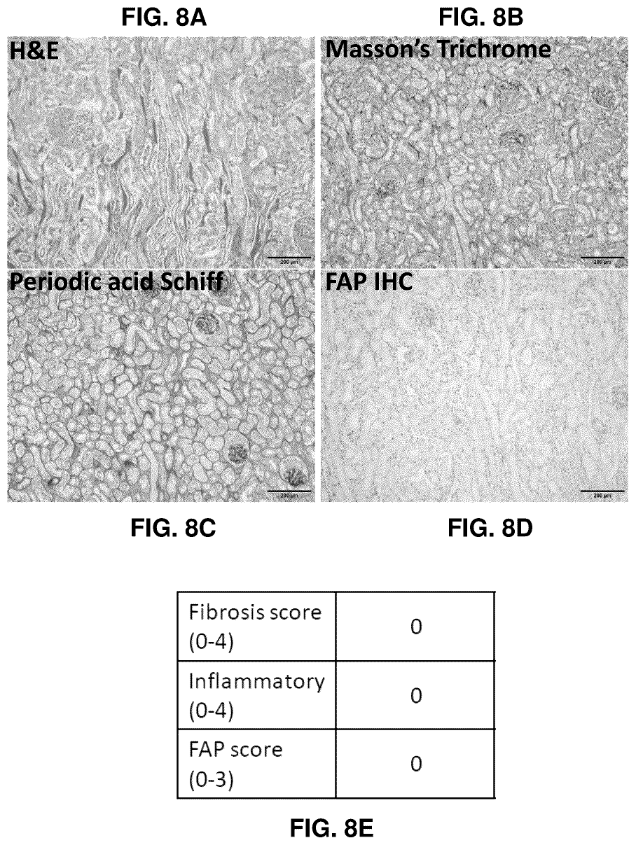

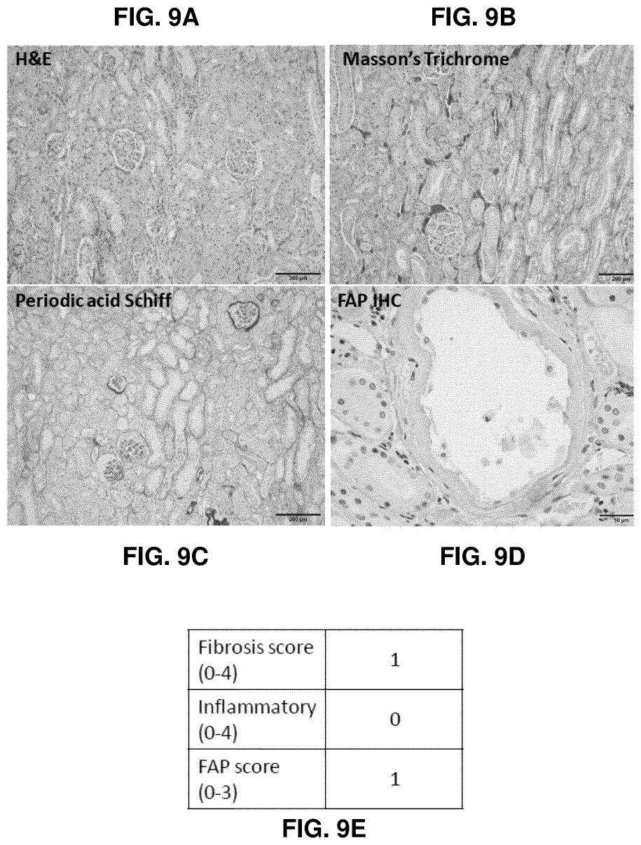

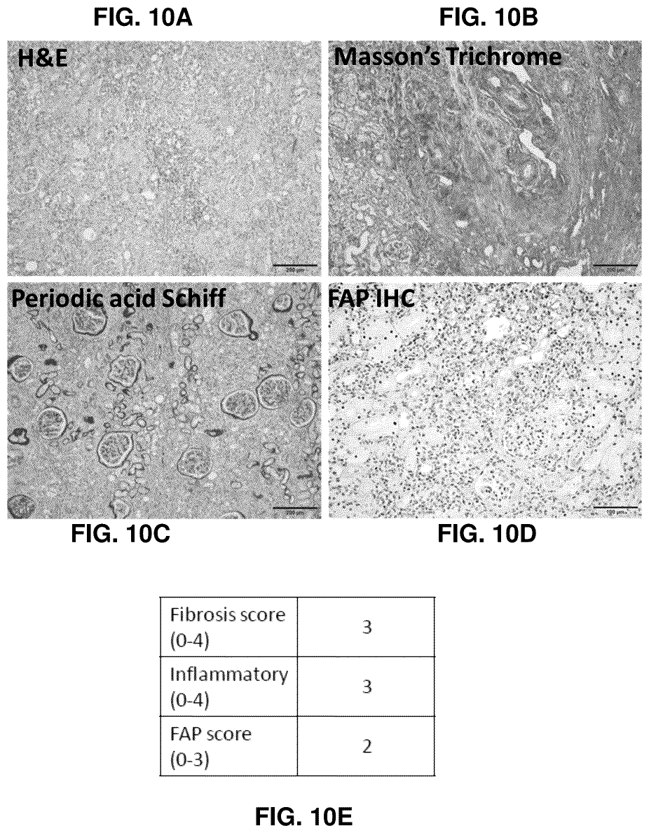

Immunohistochemistry was assessed in formalin fixed, paraffin embedded feline kidney tissue sections. For this assessment a tissue bank of 110 feline subjects were evaluated for clinical criteria to include clinical signs of chronic kidney disease (BUN/Creatinine/SDMA, imaging results, renal biopsy, gross histological description of lesions and renal morphologic description at necropsy), cause of death and age. Based on this assessment 25 feline subjects were selected for immunohistochemical analysis of feline FAP. The 25 feline subjects were selected to include young “normal” control samples, as well as cats at various stages of CKD. Normal was defined as cats younger than 8 that had no history of clinical chemistry for CKD as well as gross and histological analysis at necropsy did not note evidence of fibrosis or other sign of CKD. Sections from these subjects were processed for immunohistochemistry as well as Mason's trichrome (fibrosis staining), Periodic acid Schiff and Hematoxylin and Eosin stains. FAP was not detected in all FAP-negative tissue section samples assessed, as shown in FIGS. 8A-8D. FIG. 8E shows that the tissue sections exhibited a low FAP score, which correlated with a low fibrosis score. Even when low amounts of FAP was detected, the low FAP scores correlated with low fibrosis scores (FIGS. 10A-10E).

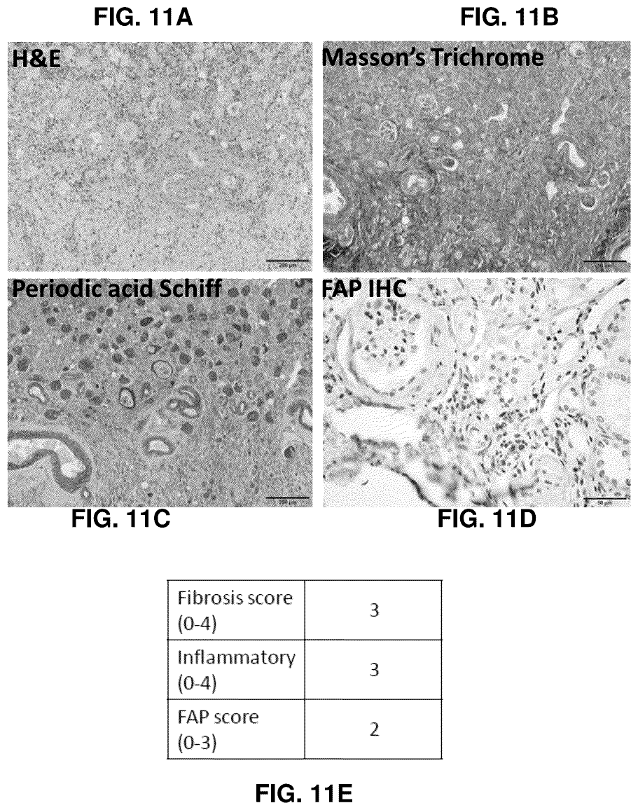

FIGS. 10A-1E and 11A-11E are exemplary of results from immunohistochemistry staining of sections from seventeen cats with renal fibrosis, showing that the majority of the cats (15 of 17) with renal fibrosis were also positive for FAP expression as shown by positive staining of the sections with FAP antibody (brown). FIGS. 10E and 11E show that the staining of tissues from two cats correlated a higher FAP score with a higher fibrosis score. Young cats with normal renal clinical chemistry and lacking histological evidence of renal disease were negative for FAP expression. Three of five older cats with normal renal clinical chemistry but evidence of renal fibrosis as demonstrated post-mortem were positive for FAP, thus demonstrating that FAP expression is useful as an early biomarker of renal disease.

The level of fibrosis from histological samples, from feline kidney, stained with Masson's trichrome stain, were performed as shown in McLeland S M et al. 2015 (McLeland S M, Cianciolo R E, Duncan C G, Quimby J M. Veterinary Pathology. 2015; 52(3):524-534). In brief; (a) Cortical scarring score of 1 with minimal tubular atrophy and interstitial expansion by increased matrix. (b) Scarring score of 2 with increased interstitial fibrosis, periglomerular fibrosis, and tubular atrophy. (c) Score of 3 with at least 50% of the tissue affected by interstitial fibrosis, tubular atrophy, and glomerulosclerosis. (d) Score of 4 with a majority of the cortical parenchyma replaced by fibrosis, tubular atrophy and loss, and glomerulosclerosis. Table 6 shows that FAP negative cats had lower fibrosis scores, indicative of a lower level of interstitial fibrosis, periglomerular fibrosis, and tubular atrophy. FAP positive cats had higher fibrosis scores, indicative of at least 50% of the tissue affected by interstitial fibrosis, tubular atrophy, and glomerulosclerosis. This data showed that the presence of FAP correlated with the presence of higher levels of fibrosis in FAP positive cats. Therefore, the method according to the present invention differentiates between stages of renal disease, especially in early stages where clinical signs are not evident, by quantifying the amount of FAP present in a sample, wherein the presence of FAP correlates with the presence of fibrosis.

TABLE 6

| Fibrosis Score | 0 | 1 | 2 | 3 | 4 | Total Cats |
|---|---|---|---|---|---|---|
| FAP Negative Cats | 8 | 2 | | | | 10 |
| FAP Positive Cats | 1 | 2 | 4 | 8 | 1 | 16 |
| Total Cats | 9 | 4 | 4 | 8 | 1 | 26 |

As is apparent to one of skill in the art, various modification and variations of the above embodiments can be made without departing from the spirit and scope of this invention. Such modifications and variations are within the scope of this invention.

All patents, publications, and patent applications cited in the present Specification are herein incorporated by reference as if each individual patent, publication, or patent application was specifically and individually indicated to be incorporated by reference in its entirety for all purposes.

What is claimed is:

1. A PCR diagnostic assay for differentially identifying early stage Chronic Kidney Disease (CDK) from late stage CDK in a subject comprising quantifying the amount of fibroblast activation protein (FAP) nucleic acid present in a sample from the subject.

2. The PCR diagnostic assay of claim 1, wherein the subject is a companion animal selected from cats, dogs, or horses.

3. The PCR diagnostic assay of claim 2, wherein the companion animal is a cat.

4. The PCR diagnostic assay of claim 1, wherein the sample is urine.

5. The PCR diagnostic assay of claim 1 wherein the nucleic acid is mRNA.

6. The PCR diagnostic assay of claim 5 wherein the mRNA is isolated from endosomes in the urine.

7. A PCR diagnostic assay for differentially identifying early stage Chronic Kidney Disease (CDK) from late stage CDK in a subject comprising:
(a) obtaining a urine sample from a subject;
(b) isolating mRNA from an endosome in the urine;
(c) using the mRNA as a template to produce cDNA by reverse transcription;
(d) amplifying the cDNA by reacting the cDNA with primers to make an amplification product; and
(e) quantitating or detecting the amplification product.

8. The PCR diagnostic assay of claim 7, wherein the subject is a companion animal selected from cats, dogs, or horses.

9. The PCR diagnostic assay of claim 8, wherein the companion animal is a cat.

10. The PCR diagnostic assay of claim 7, the primers comprise a forward primer having a nucleotide sequence as set forth in SEQ ID NO: 6 and a reverse primer having a nucleotide sequence as set forth in SEQ ID NO:7.

11. The PCR diagnostic assay of claim 10 further comprising a DNA polymerase; blocking oligonucleotides; buffers, and a detectable label.

12. The PCR diagnostic assay of claim 7, wherein the amplification product is analyzed using a probe specific for FAP to determine if FAP is present.

13. The PCR diagnostic assay of claim 12 wherein the probe includes a fluorophore as a fluorescent label.

14. The PCR diagnostic assay of claim 13, wherein the fluorophore is selected from CalO 560, CalRed 610, or FAM.

15. A PCR diagnostic assay for differentially identifying early stage Chronic Kidney Disease (CDK) from late stage CDK in a subject comprising
(a) extracting mRNA from the sample obtained from the subject;
(b) using a reverse transcriptase reaction to obtain a cDNA template;
(c) adding the cDNA, a positive control, and optionally a negative control into PCR tubes of a PCR reaction system respectively to obtain a corresponding sample reaction tube, positive reaction tube or negative reaction tube, wherein the PCR reaction system contains the primers for detecting FAP;
(d) performing a PCR reaction by placing the reaction tubes on a PCR instrument, setting parameters, and performing PCR reaction to produce an FAP amplification product;
(e) quantifying the FAP in the sample as compared to a control; wherein the presence of FAP amplification product indicates the presence of fibrosis.

16. The PCR diagnostic assay of claim 15, wherein the subject is a companion animal selected from cats, dogs, or horses.

17. The PCR diagnostic assay of claim 16, wherein the companion animal is a cat.

18. The PCR diagnostic assay of claim 15, wherein the sample is urine.
